(19) United States
(12) Patent Application Publication
(10) Pub. No.: US 2019/0071670 A1
(43) Pub. Date: Mar. 7, 2019

(54) Methods Of Treatment For Alpha-1 Antitrypsin Deficiency

(71) Applicant: Arrowhead Pharmaceuticals, Inc., Pasadena, CA (US)

(72) Inventors: Christine I. Wooddell, Madison, WI (US); Ryan M. Peterson, Madion, WI (US); Peter B. Leone, Wellesley, MA (US)

(21) Appl. No.: 16/059,708

(22) Filed: Aug. 9, 2018

Related U.S. Application Data
(63) Continuation of application No. PCT/US17/17427, filed on Feb. 10, 2017.
(60) Provisional application No. 62/293,600, filed on Feb. 10, 2016.

(51) Int. Cl. C12N 15/113; A61K 45/06; A61K 31/713; A61K 31/575; A61K 31/5415

(52) U.S. Cl. CPC C12N 15/113; A61K 45/06; A61K 31/713; C12N 2320/31; A61K 31/5415; C12N 2310/315; C12N 2310/14; A61K 31/575

(57) ABSTRACT

The application relates to methods of treatment and treatment regimens for Alpha-1 Antitrypsin deficiency (AATD) and the conditions, manifestations, and diseases caused by AATD, by the administration of one or more expression-inhibiting oligomeric compounds having a nucleobase sequence complementary to a coding sequence in the Alpha-1 Antitrypsin (A1AT or AAT) gene that inhibits the expression of the AAT gene, in combination with one or more autophagy enhancing agents that enhance and/or induce the endogenous autophagy mechanism to facilitate and encourage clearance of polymerized mutant AAT protein accumulated in the endoplasmic reticulum of hepatocytes. When used in combination, delivery of the expression-inhibiting oligomeric compounds to liver cells in vivo provides for inhibition of AAT gene expression and the use of autophagy enhancing agents increases the rate of the intracellular autophagy mechanism to clear polymerized mutant Z-AAT protein accumulated in cells, leading to an improved treatment of AATD and prevention and treatment of conditions and diseases associated with AATD.

### CROSS REFERENCE TO RELATED APPLICATIONS

This application is a continuation of International Patent Application Ser. No. PCT/US17/17427, with an international filing date of 10 Feb. 2017, designating the United States, which claims priority to U.S. Provisional Patent Application Ser. No. 62/293,600, filed 10 Feb. 2016, the contents of each of which are incorporated herein by reference in its entirety.

### SEQUENCE LISTING

This application contains a Sequence Listing which has been submitted in ASCII format and is hereby incorporated by reference in its entirety. The Sequence Listing is provided as a file named 30638_US1_SequenceListing and is 4 KB in size.

### FIELD OF THE APPLICATION

This application relates to novel methods for the treatment of Alpha-1 Antitrypsin deficiency (AATD) and conditions, manifestations, and diseases caused by AATD, comprising the administration of one or more AAT expression-inhibiting oligomeric compounds, in combination with one or more autophagy enhancing agents.

### BACKGROUND

Alpha-1 antitrypsin deficiency is an inherited autosomal codominant genetic disorder that causes defective production of alpha 1-antitrypsin leading to lung and liver diseases and occurs with a frequency of about 1 case in 1,500 to 3,500 individuals. Alpha-1 antitrypsin deficiency most often affects persons with European ancestry.

Alpha-1 Antitrypsin (al-antitrypsin, alpha-1 proteinase inhibitor, A1AT, or AAT) is a protease inhibitor belonging to the serpin superfamily. Normal AAT protein is primarily synthesized in the liver by hepatocytes and secreted into the blood. Its physiologic function is to inhibit neutrophil proteases in order to protect host tissues from non-specific injury during periods of inflammation.

The most clinically significant form of A1AT deficiency (AATD) is caused by the Z mutation. The Z mutant allele (PiZ), through a single point mutation, renders the mutant Z form protein of alpha-1 antitrypsin (referred to herein as the “Z-AAT protein”) prone to abnormal folding causing intracellular retention, as Z-AAT protein accumulates in the endoplasmic reticulum (ER) of hepatocytes. The absence of circulating anti-protease activity leaves the lung vulnerable to injury by neutrophil elastase, resulting in the development of respiratory complications such as emphysema. Weekly use of AAT augmentation therapy for AATD, using purified human AAT, results in near normal plasma levels of AAT and helps prevent lung damage in affected individuals.

While administration of purified AAT can ameliorate or prevent lung damage caused by the absence of endogenously secreted AAT, AATD patients remain vulnerable to endoplasmic reticulum liver storage disease caused by the deposition and accumulation of excessive abnormally folded AAT protein. The mutant Z-AAT protein monomers are able to form chains of ordered polymers that amass into tangled aggregates commonly referred to as “globules.” The insoluble Z-AAT protein globules are ineffective in traversing the secretory pathway and accumulate in the endoplasmic reticulum. The polymeric globule masses stress the ER. The accumulation of Z-AAT protein globules in hepatocytes is a recognized characteristic of AATD liver disease and is believed to lead to proteotoxic effects that are responsible for inducing liver injury, including liver cell damage and death and chronic liver injury, in individuals with AATD. (D. Lindblad, K. Blomenkamp and J. Teckman, Hepatology 2007, 46: 1228-1235). Patients with AATD often develop liver disease, which can be severe or fatal, even in infancy. Clinical presentations of injury in the liver include chronic hepatitis, cirrhosis, hepatocellular carcinoma, transaminitis, cholestasis, fibrosis, and even fulminant hepatic failure.

There is currently no clinically approved treatment to prevent the onset or slow the progression of liver disease caused by AATD. Because liver damage resulting from AATD occurs through a gain-of-function mechanism, inhibition of AAT gene expression may be useful in preventing accumulation of the Z-AAT protein in the liver. Further, the reduction or removal of the Z-AAT polymer aggregates may reduce the ER stress in hepatocytes, and could offer additional advantages beyond what is provided by inhibiting AAT gene expression, for the prevention and treatment of liver cell damage and chronic liver injury such as fibrosis, cirrhosis, hepatocellular carcinoma, and other conditions and diseases caused by AATD.

Expression-inhibiting oligomeric compounds with sequences designed to target the AAT gene are known to selectively and efficiently decrease expression of AAT when introduced in hepatocytes. The expression-inhibiting oligomeric compounds, such as oligonucleotides, single-stranded oligonucleotides, single stranded antisense oligonucleotides, ribozymes, double-stranded RNA (dsRNA), short interfering RNA (siRNA), micro RNAs (miRNAs), short hairpin RNAs (shRNA), dicer substrates, or other RNA-like structures that comprise nucleic acid sequences that are complementary to the mRNA of the AAT gene can inhibit expression of the AAT gene. The expression-inhibiting oligomeric compounds are capable of hybridizing to a target mRNA sequence on the AAT gene through hydrogen bonding. Various nucleotide sequences targeting the AAT gene, and various delivery vehicles and targeting molecules that successfully direct the oligomeric compounds to hepatocytes, are known in the art.

For example, Wooddell et al., in WO 2015/195628, which is incorporated herein in its entirety, discloses double-stranded nucleotide sequences comprising a sense sequence, and an antisense sequence targeted to the AAT gene, that are capable of inhibiting expression of AAT protein. As shown in WO 2015/195628, treatment with these targeted nucleotide sequences, or “RNAi triggers,” which are believed to operate through the RNA interference (RNAi) mechanism by effecting the RNA-induced silencing complex (RISC)-mediated cleavage of RNA transcripts of the AAT gene, results in significant knockdown of the AAT gene, thus decreasing the expression of mutant Z-AAT protein. Further, the double-stranded RNAi triggers used in WO 2015/195628 are shown to prevent additional Z-AAT globules from forming, greatly limiting the count, size, and area of Z-AAT globules in hepatocytes over time as compared to hepatocytes administered with saline or a control double-stranded RNAi trigger molecule unable to bind to the AAT gene.

Several other references disclose nucleotide sequences and oligonucleotide compositions that act on the RNAi pathway to inhibit expression of AAT. For example, Sehgal et al., in WO 2012/178033, which is also incorporated herein in its entirety, disclose certain sequences and delivery vehicles for double-stranded RNAi trigger molecules that may inhibit expression of the AAT gene. Additionally, Brown et al., in WO 2015/003113, also incorporated in its entirety herein, similarly disclose certain double-stranded RNAi trigger sequences and compositions that purport to reduce the expression of a target AAT gene in a cell.

Monia et al., in U.S. Patent Application Publication No. 2015/0087691, incorporated herein in its entirety, discloses oligonucleotides, single-stranded antisense compounds, and other oligomeric compounds comprising nucleotide sequences targeted to the AAT gene capable of inhibiting expression of AAT protein. According to Monia et al., the antisense oligomeric compounds disclosed in U.S. Patent Application Publication No. 2015/0087691 are believed to operate primarily through the RNase H mediated pathway, and contain targeted nucleotide sequences resulting in the purported knockdown of the AAT gene. These antisense oligonucleotides are able to decrease the expression of mutant Z-AAT protein and limit additional globules from aggregating in the endoplasmic reticulum. (See also S. Guo et al., J. Clin. Investig. 124: 251-261 (January 2014)).

With respect to Z-AAT globule accumulation, researchers have found that hepatocytes with accumulated Z-AAT globules undergo autophagy as a natural response mechanism in attempting to rid such globules. (See Teckman and Perlmutter, 279 Am. J. Physiol. Gastrointest. Liver Physiol. G961-G974 (2000)). Autophagy is a mechanism that allows for the orderly degradation and recycling of cellular components, including unnecessary or dysfunctional proteins or organelles, through a regulated process. The ability of the cell to degrade damaged and dysfunctional proteins and organelles and break them down into new amino acids for reuse by the cell is a necessary process to maintain homeostasis and for cell survival, especially in times of stress. Although the specific regulatory pathways that control autophagy are still not completely understood, it is known that autophagy is a necessary mechanism for cell survival.

Use of autophagy enhancers has been proposed as a potential approach to therapy in patients with AATD. Various researchers have found that the administration of several different classes of agents can induce or enhance the natural autophagic response in hepatocytes and reduce the hepatic load of the Z-AAT mutant protein in cells as compared to placebo. For example, Hidvegi 2010 report that the autophagy process in hepatocytes that assists in the cellular response to accumulated globules can be stimulated by treatment of the Z-AAT producing cells with the drug carbamazepine. (See T. Hidvegi et al., Science 329: 229-232 (Jul. 9, 2010)). However, along with the potentially desirable consequences of increasing autophagy, cells containing Z-AAT globules typically have higher rates of mitochondrial damage and mitochondrial autophagy, which represent negative side effects of increased autophagy. And in addition to the potential risk of increased mitochondrial damage, autophagy enhancing drugs when administered systemically may affect many other cells in the body and result in negative consequences.

A potential limitation to the use of autophagy-enhancing drugs to stimulate globule clearance is that the upregulation of autophagy due to AATD may be saturated and have limited capacity for further increase in hepatocytes with a high Z-AAT globule burden (see, e.g., Teckman et al. Am J Physiol Gastrointest Liver Physiol 283: G1156-G1165, 2002). Potential autophagy enhancing agents have been tested in vitro and/or in mice that are genetically modified to harbor the human PiZ AAT mutant allele (PiZ mice). Starvation is a stimulator of autophagy, and fasting of wild-type mice was shown to increase autophagy in the hepatocytes of these mice. In PiZ mice, however, fasting did not increase their rate of autophagy beyond that already induced by the presence of the Z-AAT protein. For at least this reason, increasing autophagy by treatment with chemical agents, either alone or in combination, may not be sustainable for the affected hepatocytes. Indeed, even at very high doses, it has been reported that various autophagy enhancing agents are unable to effectively rid Z-AAT globules from hepatocytes.

This application discloses novel methods of treatment and treatment regimens for AATD and the prevention and treatment of conditions and diseases caused by AATD, such as chronic hepatitis, cirrhosis, hepatocellular carcinoma, and fulminant hepatic failure, by the combination of expression-inhibiting oligomeric compounds that inhibit expression of the AAT gene and autophagy enhancing agents that induce and accelerate the autophagy process in hepatocytes to aid the cells in clearing accumulated Z-AAT polymeric globules.

This application further discloses novel methods of treatment and treatment regimens for AATD and the prevention and treatment of conditions and diseases caused by AATD, such as chronic hepatitis, cirrhosis, hepatocellular carcinoma, and fulminant hepatic failure, by the combination of expression-inhibiting oligomeric compounds that inhibit expression of the AAT gene and bile acid derivatives, which may optionally be administered in further combination with an autophagy enhancing agent.

### SUMMARY OF THE APPLICATION

Disclosed herein are novel methods for treating alpha-1 antitrypsin deficiency (AATD) and conditions, manifestations, or diseases caused by AATD through the administration of expression-inhibiting oligomeric compounds that selectively and efficiently decrease expression of AAT by blocking gene expression, in combination with autophagy enhancing agents that enhance the intracellular autophagy process in liver cells. The combination of AAT expression-inhibiting oligomeric compounds and autophagy enhancing agents provides a method for the therapeutic treatment of conditions and symptoms associated with AATD. Such methods of treatment disclosed in this application include administration of expression-inhibiting oligomeric compounds and autophagy enhancing agents to a mammal, including to a human.

Also disclosed herein are compositions for treating AATD and conditions and diseases caused by AATD comprising expression-inhibiting oligomeric compounds that selectively and efficiently decrease expression of AAT by blocking gene expression, and autophagy agents that enhance the intracellular autophagy process in liver cells. The compositions comprising AAT expression-inhibiting oligomeric compounds and autophagy enhancing agents disclosed herein may be administered to provide for the therapeutic treatment of diseases associated with AATD to a mammal, including to a human.

Disclosed herein are methods for the treatment of AATD comprising administering to a subject an AAT expression-inhibiting oligomeric compound in combination with an autophagy enhancing agent. The AAT expression-inhibiting oligomeric compound may be administered separately from or in the same composition as the autophagy enhancing agent.

Disclosed herein are methods for the treatment of AATD comprising administering to a human patient in need of such treatment an AAT expression-inhibiting oligomeric compound in combination with an autophagy enhancing agent. The AAT expression-inhibiting oligomeric compound may be administered separately from or in the same composition as the autophagy enhancing agent.

Disclosed herein are methods for the treatment of AATD comprising administering to a human patient in need of such treatment a composition comprising an AAT expression-inhibiting oligomeric compound in combination with a composition comprising an autophagy enhancing agent. The composition comprising an AAT expression-inhibiting oligomeric compound may be administered separately from or in the same composition as the autophagy enhancing agent.

Disclosed herein are methods for the treatment of AATD comprising administering to a patient in need of such treatment an AAT expression-inhibiting oligomeric compound in combination with an autophagy enhancing agent, wherein inhibiting expression of AAT gene in an organism combined with increasing the intracellular autophagic response treats, prevents, or manages a pathological condition or disease caused by alpha-1 antitrypsin deficiency.

Disclosed herein are methods for the treatment of AATD, comprising administering to a patient in need of such treatment an AAT expression-inhibiting oligomeric compound in combination with an autophagy enhancing agent, wherein inhibiting expression of AAT gene in an organism combined with increasing the intracellular autophagic response treats, prevents, or manages a pathological condition or disease caused by alpha-1 antitrypsin deficiency consisting of: chronic hepatitis, cirrhosis, hepatocellular carcinoma, and fulminant hepatic failure.

Disclosed herein are methods for the treatment of alpha-1 antitrypsin deficiency (AATD) comprising administering to a patient in need of such treatment an AAT expression-inhibiting oligomeric compound in combination with an autophagy enhancing agent, and further comprising a second AAT expression-inhibiting oligomeric compound.

Disclosed herein are methods for the treatment of AATD, comprising administering to a patient in need of such treatment an AAT expression-inhibiting oligomeric compound, wherein the AAT expression-inhibiting oligomeric compound has a nucleobase sequence that contains at least one modified nucleotide or modified internucleoside linkage, in combination with an autophagy enhancing agent.

Disclosed herein are methods for the treatment of AATD, comprising administering to a subject an AAT expression-inhibiting oligomeric compound in combination with an autophagy enhancing agent, and further administering a second autophagy enhancing agent.

Disclosed herein are methods for the treatment of AATD comprising administering to a subject an expression-inhibiting oligomeric compound that has a nucleobase sequence complementary to a coding sequence in the Alpha-1 Antitrypsin gene wherein the expression-inhibiting oligomeric compound is a double-stranded RNAi agent, in combination with an autophagy enhancing agent.

Disclosed herein are methods for the treatment of AATD, comprising administering to a patient in need of such treatment an AAT expression-inhibiting oligomeric compound having one or more modified nucleotides, and wherein the expression-inhibiting oligomeric compound is a double-stranded RNAi agent, in combination with an autophagy enhancing agent.

Disclosed herein are methods for the treatment of AATD, comprising administering an AAT expression-inhibiting oligomeric compound that comprises at least 15 consecutive nucleotides of any of the sequences of SEQ ID NOS. 1-6, in combination with an autophagy enhancing agent.

Disclosed herein are methods for the treatment of AATD, comprising administering an AAT expression-inhibiting oligomeric compound that comprises at least 16 consecutive nucleotides of any of the sequences of SEQ ID NOS. 1-6, in combination with an autophagy enhancing agent.

Disclosed herein are methods for the treatment of AATD, comprising administering an AAT expression-inhibiting oligomeric compound that comprises any of the sequences of SEQ ID NOS. 1-6, in combination with an autophagy enhancing agent.

Disclosed herein are methods for the treatment of AATD, comprising administering to subject an effective amount of an AAT expression-inhibiting oligomeric compound and an effective amount of an autophagy enhancing agent that is identified in Table 4.

Disclosed herein are methods for the treatment of AATD, comprising administering to subject an effective amount of an AAT expression-inhibiting oligomeric compound and an effective amount of an autophagy enhancing agent, wherein the autophagy enhancing agent is flu phenazine.

Disclosed herein are methods for the treatment of AATD, comprising administering to subject an effective amount of an AAT expression-inhibiting oligomeric compound and an effective amount of an autophagy enhancing agent, wherein the autophagy enhancing agent is a bile acid derivative.

Disclosed herein are methods for the treatment of AATD, comprising administering to a subject an AAT expression-inhibiting oligomeric compound, wherein the expression-inhibiting oligomeric compound is further conjugated to a targeting moiety, in combination with an autophagy enhancing agent.

Disclosed herein are methods for the treatment of AATD, comprising administering to a subject an AAT expression-inhibiting oligomeric compound, wherein the expression-inhibiting oligomeric compound is further conjugated to a targeting moiety, in combination with an autophagy enhancing agent. In some embodiments, the targeting moiety comprises an asialoglycoprotein receptor ligand. In some embodiments, the targeting moiety comprises an asialoglycoprotein receptor ligand that comprises a galactose derivative. In some embodiments, the targeting moiety comprises an N-acetylgalactosamine (GalNAc). In some embodiments, the targeting moiety comprises a GalNAc cluster. In some embodiments, the targeting moiety comprises a GalNAc trimer or GalNAc tetramer.

Disclosed herein are methods for the treatment of AATD, comprising administering to a subject an AAT expression-inhibiting oligomeric compound, wherein the expression-inhibiting oligomeric compound is a double-stranded RNAi agent that is further conjugated to a targeting moiety, in combination with an autophagy enhancing agent. In some embodiments, the targeting moiety is conjugated, directly or indirectly (e.g., via a linking group), to the sense strand of the double-stranded RNAi agent. In some embodiments, the targeting moiety is conjugated to the 5′ terminal end of the sense strand of the double stranded RNAi agent.

Disclosed herein are methods for the treatment of AATD, comprising administering to a subject an effective amount of an AAT expression-inhibiting oligomeric compound that is a single-stranded oligonucleotide, in combination with an effective amount of an autophagy enhancing agent.

Disclosed herein are methods for the treatment of AATD, comprising administering to subject an effective amount of AAT expression-inhibiting oligomeric compound that is a single-stranded antisense oligonucleotide, in combination with an effective amount of an autophagy enhancing agent.

Disclosed herein are kits for use in the treatment AATD, said kits comprising a composition comprising an AAT expression-inhibiting oligomeric compound and a second composition comprising an autophagy enhancing agent.

Disclosed herein are methods for the treatment of AATD, comprising administering to a subject an AAT expression-inhibiting oligomeric compound in combination with a bile acid derivative and in further combination with another autophagy enhancing agent.

Disclosed herein are methods for reducing Z-AAT globule burden (also referred to as Z-AAT polymeric protein) in a liver cell, comprising administering to the liver cell an effective amount of an AAT expression-inhibiting oligomeric compound, or a composition comprising same, and an effective amount of an autophagy enhancing agent, or a composition comprising same. In some embodiments, the liver cell is a hepatocyte.

It should be understood that the detailed description and figures, while disclosing some embodiments, is not intended to be limiting. Various changes and modifications within the scope of the invention will be apparent to those of ordinary skill in the art. Other features and advantages of the invention will be apparent from the following detailed description, and from the claims.

### BRIEF DESCRIPTION OF THE DRAWINGS

FIG. 1. Graph depicting relative serum AAT levels in PiZ mice treated saline or Duplex Pair SEQ ID NO: 5/6 AAT RNAi agent (referred to as AD00370) with Melittin-like peptide (MLP) delivery polymer.

FIG. 2. PAS-D staining to visualize Z-AAT accumulation in liver. Liver sections from (A) PiZ mouse sacrificed at day 1 of study; (B) PiZ mouse that received four biweekly intravenous (IV) doses of saline vehicle; (C) PiZ mouse that received four biweekly IV doses of 8 mg/kg Luciferase siRNA control with 8 mg/kg MLP delivery polymer; (D) PiZ mouse that received four biweekly IV doses of 8 mg/kg Duplex Pair SEQ ID NO: 5/6 with 8 mg/kg of MLP delivery polymer.

FIG. 3. PiZ mice initially 5 weeks old that received four biweekly IV doses of either saline vehicle (n=7) or 8 mg/kg Duplex Pair SEQ ID NO: 5/6 AAT RNAi agent (AAT-UNA) with 8 mg/kg of MLP delivery polymer (n=10). The livers were sectioned, processed in formalin, and stained with PAS-D for digital quantitation of the globule number, size and the area of the liver covered by globules.

FIG. 4A. Soluble and insoluble fractions from livers of PiZ, mice analyzed by semi-quantitative Western blot with anti-hAAT antibody. Five-week old mice received four biweekly IV doses of saline, 8 mg/kg Luciferase siRNA control (LUC-UNA/Duplex Pair SEQ ID NO: 7/8) with 8 mg/kg of MLP delivery polymer, or 8 mg/kg Duplex Pair SEQ ID NO: 5/6 AAT RNAi agent (AAT-UNA) with 8 mg/kg of MLP delivery polymer for 8 weeks.

FIG. 4B. Bar graph showing quantitation of Z-AAT monomer and polymer content in livers of PiZ mice. Five-week old mice received four biweekly IV doses of saline vehicle (n=7), 8 mg/kg Luciferase siRNA control (LUC-UNA/Duplex Pair SEQ ID NO: 7/8) with 8 mg/kg of MLP delivery polymer (n=3), or 8 mg/kg Duplex Pair SEQ ID NO: 5/6 AAT RNAi agent (AAT-UNA) with 8 mg/kg of MLP delivery polymer (n=10) for 8 weeks.

FIG. 5. Bar graph showing percentage area of liver covered by globules and globule size measured in six month old female PiZ mice that received a single IV dose of saline, 8 mg/kg Luciferase siRNA control (LUC-UNA/Duplex Pair SEQ ID NO: 7/8) with 8 mg/kg of MLP delivery polymer, or 8 mg/kg Duplex Pair SEQ ID NO: 5/6 AAT RNAi agent (AAT-UNA) with 8 mg/kg of MLP delivery polymer. The livers were sectioned, processed in formalin for histological observation, and stained with PAS-D for digital quantitation of the globule size and the area of the liver covered by globules.

FIG. 6. Graph showing AAT knockdown following repeat administration in primates with Duplex Pair SEQ ID NO: 5/6 AAT RNAi agent (AAT-UNA) and MLP delivery polymer. Two monkeys each were given 2.0 mg/kg MLP delivery polymer (MLP delivery peptide)+4.0 Duplex Pair SEQ ID NO: 5/6 AAT RNAi agent (AAT-UNA) or 3 mg/kg MLP delivery polymer (MLP delivery peptide)+6 mg/kg Duplex Pair SEQ ID NO: 5/6 AAT RNAi agent (AAT-UNA). The first dose was at day 1. Doses were all six weeks apart.

FIG. 7. Western blot analysis of the soluble (Monomer) and insoluble (Polymer) fractions of Z-AAT from livers of PiZ mice, and bar graph showing levels of Z-AAT monomer from the soluble fraction and Z-AAT polymer from the insoluble fraction, shown as relative percent change compared to baseline. Male PiZ mice that were 11-17 weeks old were treated biweekly with 8 mg/kg ARC-AAT (8 mg/kg Duplex Pair SEQ ID NO: 5/6 AAT RNAi agent+4 mg/kg MLP delivery peptide) for 32 or 33 weeks. These were compared with mice euthanized at the start of study (baseline) and control mice injected for 32 or 33 weeks with saline. Monomeric and polymeric Z-AAT protein in the livers of these mice were compared by semi-quantitative Western blotting. GAPDH is shown as a loading control.

FIG. 8. Hematoxylin and eosin staining to visualize liver morphology as a result of treatment with 8 mg/kg ARC-AAT (8 mg/kg Duplex Pair SEQ ID NO: 5/6 AAT RNAi agent+4 mg/kg MLP delivery peptide). Male PiZ mice that were 11-17 weeks old were treated biweekly (q2w) with ARC-AAT at doses of 8 mg/kg for 33 weeks. Liver histology was compared between mice at start of study (baseline), control mice injected at the same intervals with saline, and mice injected with ARC-AAT. Black arrows indicate examples of compressed nuclei. Light-colored arrows indicate inflammatory cell infiltration.

FIG. 9. Western blot analysis of the soluble and insoluble fractions of Z-AAT from livers of PiZ mice. Male PiZ mice that were 15-21 weeks old were treated biweekly with 8 mg/kg ARC-AAT (8 mg/kg Duplex Pair SEQ ID NO: 5/6 AAT RNAi agent+4 mg/kg MLP delivery peptide) for 38 weeks. These were compared with mice at start of study (baseline) and control mice injected for 38 weeks with saline vehicle. Monomeric and polymeric Z-AAT protein in the livers of these mice were compared by semi-quantitative Western blotting. GAPDH is shown as a loading control.

### DETAILED DESCRIPTION

Disclosed herein are methods of treatment for AATD comprising administering to a subject an effective amount of a composition comprising one or more AAT expression-inhibiting oligomeric compounds in combination with administering to the same subject an effective amount of a composition comprising one or more autophagy enhancing agents. The AAT expression-inhibiting oligomeric compounds inhibit the expression of AAT in a cell, group of cells, tissue, or subject, and the autophagy enhancing agents induce, enhance and/or accelerate the intracellular autophagy process and facilitate the removal and elimination of Z-AAT globules or portions of the globules retained in hepatocytes, leading to an improved treatment of AATD and prevention and treatment of conditions and diseases associated with AATD.

Furthermore, the application relates to methods for inhibiting expression of the AAT gene in a cell, tissue or organism while simultaneously reducing the count, size, and/or area percentage of Z-AAT globules retained in the cell, tissue or organism, comprising the steps of: introducing into the cell, tissue or organism an expression-inhibiting oligomeric compound as defined herein; introducing into the cell, tissue or organism an autophagy enhancing agent as defined herein; maintaining said cell, tissue or organism for a time sufficient to obtain degradation of the mRNA transcript of AAT, thereby inhibiting expression of AAT in a given cell; and maintaining said cell, tissue or organism for a time sufficient to obtain degradation of the accumulated Z-AAT globules.

The present application discloses the combination of expression-inhibiting oligomeric compounds directed to AAT with one or more autophagy enhancing agents, leading to benefits and advantages for the treatment of AATD over previously known treatments and treatment regimens. The ability to significantly and promptly reduce or eliminate the Z-AAT globule burden can provide benefits beyond those provided by inhibiting expression of the Z-AAT protein alone, as it can serve to change the capacity of the subject's hepatocytes to engage in autophagy for further clearance of the polymers.

The methods disclosed herein envisage that the AAT expression-inhibiting oligomeric compounds and the autophagy enhancing agents may be administered in the same dosage form or in different dosage forms. The methods disclosed herein also envisage that the AAT expression-inhibiting oligomeric compounds and the autophagy enhancing agents may be administered according to the same route of administration or different routes of administration. For example, in some embodiments, an AAT expression-inhibiting oligomeric compound and an autophagy enhancing agent are both administered parenterally. In other embodiments, for example, an AAT expression-inhibiting oligomeric compound is administered parenterally, and an autophagy enhancing agent is administered orally.

The methods disclosed herein envisage that the AAT expression-inhibiting oligomeric compounds and the autophagy enhancing agents may be administered in the same pharmaceutical composition or in different pharmaceutical compositions. For example, in some embodiments, an AAT expression-inhibiting oligomeric compound and an autophagy enhancing agent are both administered parenterally in the same pharmaceutical composition cocktail. In some embodiments, an AAT expression-inhibiting oligomeric compound and an autophagy enhancing agent are both administered parenterally but in separate pharmaceutical compositions. In some embodiments, an AAT expression-inhibiting oligomeric compound and an autophagy enhancing agent are administered sequentially, but are administered in different dosage forms and in separate pharmaceutical compositions.

The AAT expression-inhibiting oligomeric compounds and the autophagy agents may be administered at the same time or they may be administered at different times. For example, treatment with an autophagy enhancing agent may be given prior to, concomitant with, or following treatment with an expression-inhibiting oligomeric compound. In some embodiments, an autophagy enhancing agent is administered prior to administration of an expression-inhibiting oligomeric compound. In other embodiments, an autophagy enhancing agent is administered after administration of an expression-inhibiting oligomeric compound. In some embodiments, an autophagy enhancing agent and an expression-inhibiting oligomeric compound are administered simultaneously or near-simultaneously.

In some embodiments, an AAT expression-inhibiting oligomeric compound and an autophagy enhancing agent are each administered once. In other embodiments, administration of an AAT expression-inhibiting oligomeric compound is repeated (i.e., repeat-dose regimen or multi-dose regimen), and an autophagy enhancing agent is administered once. In other embodiments, administration of an AAT expression-inhibiting oligomeric compound is repeated (i.e., repeat-dose regimen or multi-dose regimen), and administration of an autophagy enhancing agent is repeated (i.e., repeat-dose regimen or multi-dose regimen). For repeated administrations, an AAT expression-inhibiting oligomeric compounds may be administered to the subject once daily, every other day, once every three days, once every four days, once every five days, once every six days, twice a week, once a week, once every two weeks, once every three weeks, once every four weeks, once every four (4) to fourteen (14) weeks, twice a month, once a month, once every two months, once every three months, or once every four months or longer. For repeated administrations, autophagy enhancing agents may be administered to the subject once daily, twice daily, three times daily, four times daily, every other day, once every three days, once every four days, once every five days, once every six days, twice a week, once a week, once every two weeks, once every three weeks, once every four weeks, once every four (4) to fourteen (14) weeks, twice a month, once a month, once every two months, once every three months, or once every four months or longer. Values intermediate to the recited values are also intended to be part of this invention.

In some embodiments, AAT expression-inhibiting oligomeric compounds are administered as necessary. In some embodiments, autophagy enhancing agents are administered as necessary. In some embodiments, both the AAT expression-inhibiting oligomeric compounds and the autophagy enhancing agents can be administered as necessary.

In some embodiments, an initial treatment regimen for an AAT expression-inhibiting oligomeric compound and/or an autophagy enhancing agent may comprise repeat administration at an initial time interval and subsequent administration on a less frequent basis. For example, in some embodiments, after administration of an AAT expression-inhibiting oligomeric compound weekly or biweekly for one to six months, administration can thereafter be repeated once per month or less. The initial time interval can be a set number of administrations, a set span of time, or until a determined reduction in AAT is measured. Similarly, in some embodiments, after administration of an autophagy enhancing agent on a daily, twice daily, three times daily, four times daily, every other day, bi-weekly, or weekly basis for one to six months, administration can thereafter be repeated on a less frequent basis. In some embodiments, an AAT expression-inhibiting oligomeric compound may be administered on a set interval for one to six months, after which an autophagy enhancing agent may be administered in combination with an AAT expression-inhibiting oligomeric compound for a set time interval. In some embodiments, after administration of an autophagy enhancing agent on a daily, twice daily, three times daily, four times daily, every other day, bi-weekly, or weekly basis for one to six months, in combination with the administration of an AAT expression-inhibiting oligomeric compound, administration with an autophagy agent is no longer necessary, as the patient has removed or sufficiently eliminated or removed the accumulated Z-AAT globules to reduce any proteotoxic effects and the subject is able to maintain relatively low levels of Z-AAT globules without the need for continued administration of an autophagy enhancing agent. The initial time interval for administering an autophagy enhancing agent can be a set number of administrations, a set span of time, or until a determined reduction in Z-AAT globules is measured. For any dosing regimen, whether single or repeat, any of the above amounts may be used. For repeat dosing, the same dose or different doses may be used for each administration.

In some embodiments, the treatment regimen may comprise administering an AAT expression-inhibiting oligomeric compound for one week prior to the first administration of an autophagy enhancing agent. In some embodiments, administration of an AAT expression-inhibiting oligomeric compound may be given for months prior to the first administration of an autophagy enhancing agent. In some embodiments, an autophagy enhancing agent may be administered for just a few days prior to, during, or following treatment with an AAT expression-inhibiting oligomeric compound. In some embodiments, an autophagy enhancing agent may be administered as a long-term treatment prior to, during, or following treatment with an AAT expression-inhibiting oligomeric compound. In some embodiments, an AAT expression-inhibiting oligomeric compound may be administered as a long-term treatment prior to, during, or following treatment with an autophagy enhancing agent.

The particular AAT expression-inhibiting oligomeric compounds to be used and the optimal dose level for any patient will depend on a variety of factors including the efficacy of the specific oligomeric compounds employed, the age, body weight, physical activity, diet, and overall health of the patient, as well as the severity of the case. Similarly, the particular autophagy enhancing agents to be used and the optimal dose level for any patient will depend on a variety of factors including the efficacy of the specific autophagy enhancing agents employed, the age, body weight, physical activity, diet, and overall health of the patient, as well as the severity of the case.

In some embodiments, one AAT expression-inhibiting oligomeric compound may be administered. In some embodiments, one autophagy enhancing agent may be administered. In some embodiments, more than one AAT expression-inhibiting oligomeric compound may be administered. In some embodiments, more than one autophagy enhancing agent may be administered.

In some embodiments, the administration of an autophagy enhancing agent enhances the effect of an AAT expression-inhibiting oligomeric compound, such that the resulting effect is greater than the effect of administering the AAT expression-inhibiting oligomeric compound alone. In some embodiments, the administration of an AAT expression-inhibiting oligomeric compound enhances the effect of the autophagy enhancing agent, such that the resulting effect is greater than the effect of administering an autophagy enhancing agent alone. In some embodiments, the co-administration of AAT expression-inhibiting oligomeric compounds and autophagy enhancing agents results in an effect that is additive of the effects of the compounds when administered alone. In some embodiments, the co-administration of the autophagy enhancing agents may improve the effect relative to administering an expression-inhibiting oligomeric compound alone. In other embodiments, a synergistic effect is experienced when combining the administration of an AAT expression-inhibiting oligomeric compound and an autophagy enhancing agent.

The AAT expression-inhibiting oligomeric compounds and the autophagy enhancing agents may be packaged separately or included together in a kit, container, pack, or dispenser. The AAT expression-inhibiting oligomeric compounds and/or the autophagy enhancing agents may be packaged in pre-filled syringes or vials.

The herein described methods also provide for cells comprising at least one of the AAT expression-inhibiting oligomeric compounds, in combination with a separately administered autophagy enhancing agent. In some embodiments, the cell is preferably a mammalian cell, such as a human cell. Furthermore, tissues and/or non-human organisms comprising expression-inhibiting oligomeric compounds and autophagy enhancing agents are an embodiment of this invention, whereby said tissues and/or non-human organisms are particularly useful for research purposes or as research tools, for example in drug testing.

The methods disclosed herein include that the subject is administered a therapeutically effective amount of any one or more of the described AAT expression-inhibiting oligomeric compounds and any one or more of the autophagy enhancing agents. Treatment of a subject that would benefit from a reduction and/or inhibition of AAT gene expression and a reduction and/or elimination of Z-AAT protein globules retained in hepatocytes includes therapeutic and/or prophylactic treatment in individuals that express mutant Z-AAT protein. The subject can be a mammal including a human.

In some embodiments, the disclosed methods provide for treating, preventing or managing clinical presentations associated with AATD. In some embodiments, the disclosed methods comprise administering to a subject in need of such treatment (including but not limited to a subject in need of the prevention and/or management of the manifestations or ailments associated with AATD) a therapeutically or prophylactically effective amount of one or more of the AAT expression-inhibiting oligomeric compounds described herein in combination with one or more of the autophagy enhancing agents described herein. Preferably, said subject is a mammal, most preferably a human patient.

### Definitions

As used herein, “expression-inhibiting oligomeric compounds” include, but are not limited to: oligonucleotides, single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. Expression-inhibiting oligomeric compounds comprise nucleotide sequences containing 10-50 nucleotides and having a nucleobase sequence complementary to a coding sequence in an expressed target gene within the cell. For example, AAT expression-inhibiting oligomeric compounds generally have a nucleobase sequence complementary to a coding sequence in the Alpha-1 Antitrypsin gene. The expression-inhibiting oligomeric compounds that are the subject of this application, upon delivery to a cell expressing the AAT gene, inhibit the expression of said AAT gene. The AAT gene expression can be inhibited in vitro or in vivo. Specific sequences of expression-inhibiting oligomeric compounds that are able to knockdown the AAT gene are known in the art.

The term “single-stranded oligonucleotide,” as used herein, means a single-stranded oligomeric compound having a sequence at least partially complementary to a target mRNA, that is capable of undergoing hybridization to a target mRNA through hydrogen bonding. In some embodiments, a single-stranded oligonucleotide is a single stranded antisense oligonucleotide.

As used herein, the term “oligonucleotide” means a polymer of linked nucleosides each of which can be independently modified or unmodified.

As used herein, an “RNAi agent” refers to an agent that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein, an RNAi agent is a type of an expression-inhibiting oligomeric compound. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents may include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), and dicer substrates. The RNAi agents described herein are comprised of an oligonucleotide having a strand that is at least partially complementary to the mRNA being targeted. In some embodiments, the RNAi agents described herein are double-stranded, and are comprised of an antisense strand, and a sense strand that is at least partially complementary to the antisense strand. Double-stranded RNAi agents typically have a duplex length in the range of about 16 to 30 nucleotides, and are commonly comprised of an antisense strand comprising 17, 18, 19, 20, 21, 22, 23, 24, 25, or 26 nucleotides and a sense strand comprising 17, 18, 19, 20, 21, 22, 23, 24, 25, or 26 nucleotides.

The sense and antisense strands can be the same length or they can be different lengths. As an example, a sense strand can be 21 nucleotides in length while an antisense strand can be 23 nucleotides in length. RNAi agents may be comprised of modified nucleotides and/or one or more non-phosphodiester linkages. In some embodiments, the RNAi agents described herein are single-stranded oligonucleotides.

“Silence”, “reduce”, “inhibit”, “down-regulate”, or “knockdown” gene expression, in as far as they refer to an AAT gene, means that the expression of the gene, as measured by the level of RNA transcribed from the gene or the level of polypeptide, protein or protein subunit translated from the mRNA in a cell, group of cells, tissue, or subject in which the AAT gene is transcribed, is reduced when the cell, group of cells, tissue, or subject is treated with AAT expression-inhibiting oligomeric compounds described herein as compared to a second cell, group of cells, tissue, or subject but which has or have not been so treated.

As used herein, the term “sequence” or “nucleotide sequence” refers to a succession or order of nucleobases or nucleotides, described with a succession of letters using the standard nucleotide nomenclature.

As used herein, and unless otherwise indicated, the term “complementary,” when used to describe a first nucleotide sequence (e.g., double-stranded AAT RNA agent sense strand or AAT mRNA) in relation to a second nucleotide sequence (e.g., single-stranded AAT antisense oligonucleotide or a double-stranded AAT RNAi agent antisense strand), refers to the ability of an oligonucleotide or polynucleotide comprising the first nucleotide sequence to hybridize (form base pair hydrogen bonds) and form a duplex or double helical structure under certain conditions with an oligonucleotide or polynucleotide comprising the second nucleotide sequence. Complementary sequences include Watson-Crick base pairs or non-Watson-Crick base pairs and include natural or modified nucleotides or nucleotide mimics, at least to the extent that the above requirements with respect to the ability to hybridize are fulfilled. Perfectly or fully complementary means that all (100%) of the bases in a contiguous sequence of a first polynucleotide will hybridize with the same number of bases in a contiguous sequence of a second polynucleotide. The contiguous sequence may comprise all or a part of a first or second nucleotide sequence. As used herein, partially complementary means that in a hybridized pair of nucleobase sequences, at least 70% of the bases in a contiguous sequence of a first polynucleotide will hybridize with the same number of bases in a contiguous sequence of a second polynucleotide. As used herein, substantially complementary means that in a hybridized pair of nucleobase sequences, at least 85% of the bases in a contiguous sequence of a first polynucleotide will hybridize with the same number of bases in a contiguous sequence of a second polynucleotide. The terms “complementary”, “fully complementary” and “substantially complementary” herein may be used with respect to the base matching between the sense strand and the antisense strand of a double-stranded AAT RNAi agent, between the antisense strand of a double-stranded AAT RNAi agent and a portion of AAT mRNA, or between a single-stranded AAT antisense oligonucleotide and a portion of AAT mRNA.

The terms “treat”, “treatment”, and the like, mean in context of this invention the relief from or alleviation of a disorder related to AATD.

“Introducing into a cell”, when referring to an expression-inhibiting oligomeric compound, means functionally delivering the expression-inhibiting oligotnefic compound into a cell. By functional delivery, it is meant that the expression-inhibiting oligomeric compound is delivered to the cell and has the expected biological activity, sequence-specific inhibition of gene expression. Many molecules, including expression-inhibiting oligomeric compounds, administered to the vasculature of a mammal are normally cleared from the body by the liver. Clearance of an expression-inhibiting oligomeric compound by the liver wherein the expression-inhibiting oligomeric compound is degraded or otherwise processed for removal from the body and wherein the expression-inhibiting oligomeric compound does not cause sequence-specific inhibition of gene expression is not considered functional delivery.

“Therapeutically effective amount,” as used herein, when referring to an AAT expression-inhibiting oligomeric compound, is intended to include the amount of an AAT expression-inhibiting oligomeric compound, that, when administered to a subject having a AATD, is sufficient to effect treatment of the disease (e.g., by diminishing, ameliorating or maintaining the existing disease or one or more symptoms of disease). The “therapeutically effective amount” may vary depending on the AAT expression-inhibiting oligomeric compound, any co-treatment administered, how the compounds are administered, the disease and its severity and the history, age, weight, family history, genetic makeup, the types of preceding or concomitant treatments, if any, and other individual characteristics of the subject to be treated.

“Therapeutically effective amount,” as used herein, when referring to an autophagy enhancing agent, is intended to include the amount of an autophagy enhancing agent, that, when administered to a subject having a AATD, is sufficient to effect treatment of the disease (e.g., by diminishing, ameliorating or maintaining the existing disease or one or more symptoms of disease). The “therapeutically effective amount” may vary depending on the autophagy enhancing agent, any co-treatment administered, how the agents are administered, the disease and its severity and the history, age, weight, family history, genetic makeup, the types of preceding or concomitant treatments, if any, and other individual characteristics of the subject to be treated.

As used herein, “pharmacologically effective amount,” “therapeutically effective amount,” or simply “effective amount” refers to that amount of an AAT expression-inhibiting oligomeric compound and/or autophagy enhancing agent to produce the intended pharmacological, therapeutic or preventive result. “Prophylactically effective amount,” as used herein, is intended to include the amount of an AAT expression-inhibiting oligomeric compound and/or an autophagy enhancing agent, that, when administered to a subject having a AATD but not yet (or currently) experiencing or displaying symptoms of the disease, and/or a subject at risk of developing a AATD, is sufficient to prevent or minimize the severity of the disease or one or more symptoms of the disease. Minimizing the severity of the disease includes slowing the course of the disease. As used herein, a “prophylactically effective amount” is considered to be a species of a “therapeutically effective amount.”

As used herein, a “pharmaceutical composition” when referring to an AAT expression-inhibiting oligomeric compound comprises a pharmacologically effective amount of at least one AAT expression-inhibiting oligomeric compound and a pharmaceutically acceptable carrier and optionally one or more a pharmaceutically acceptable excipients. As used herein, a “pharmaceutical composition” when referring to an autophagy enhancing agent comprises a pharmacologically effective amount of at least one autophagy enhancing agent and a pharmaceutically acceptable carrier and optionally one or more a pharmaceutically acceptable excipients.

### Expression-Inhibiting Oligomeric Compounds

The AAT expression-inhibiting oligomeric compounds described herein may be comprised of naturally occurring nucleotides or may be comprised of one or more modified nucleotides or nucleotide mimics. The expression-inhibiting oligomeric compounds may be synthesized and/or modified by methods well established in the art. Exemplary sequences of double stranded AAT expression-inhibiting oligomeric compounds are shown in the following Tables 1, 2, 3A, and 3B:

TABLE 1
Exemplary double-stranded AAT RNAi trigger molecule core sequences.

| | | | | Duplex |
|---|---|---|---|---|
| SEQ | | SEQ | | Pair |
| ID | Antisense Strand Sequence | ID | Sense Strand Sequence | SEQ ID |
| NO: | (5′→3′) | NO: | (5′→3′) | NO: |
| 1 | GGAACUUGGUGAUGAUAU | 2 | AUAUCAUCACCAAGUUCC | 1/2 |

TABLE 2
Exemplary unmodified double-stranded AAT RNAi trigger molecule sequences.

| | | | | Duplex |
|---|---|---|---|---|
| SEQ | | SEQ | | Pair |
| ID | Antisense Strand Sequence | ID | Sense Strand Sequence | SEQ ID |
| NO: | (5′→3′) | NO: | (5′→3′) | NO: |
| 3 | TGGAACUUGGUGAUGAUAUTT | 4 | UAUAUAUCAUCACCAAGUUCCAT | 3/4 |

TABLE 3A
Exemplary modified double-stranded AAT RNAi
trigger molecule antisense strand sequences.

| SEQ ID NO: | Antisense Strand Sequence (5′→3′) |
|---|---|
| 5 | dTGfgAfaCfUᵤₙₐUfgGfuGfaUfgAfuAfudTsdT |

TABLE 3B
Exemplary modified double-stranded AAT RNAi
trigger molecule sense strand sequences.

| SEQ ID | |
|---|---|
| NO: | Sense Strand Sequence (5′→3′) |
| 6 | (Chol-TEG)uAuAfuAfuCfaUfcAfcCfaAfgUfuCfcAf |
| | (invdT) |

In Tables 3A and 3B, the following notations are used to indicate modified nucleotides, targeting groups and linking groups:
N=2′-OH (unmodified) ribonucleotide (capital letter without for d indication)
n=2′-OMe modified nucleotide
Nf=2′-fluoro modified nucleotide
dN=2′-deoxy nucleotides
N_{UNA}=2′,3′-seco nucleotide mimics (unlocked nucleobase analogs)
(invdN)=inverted deoxyribonucleotide (3′-3′ linked nucleotide)
s=phosphorothioate linked nucleotide

In Tables 3A and 3B, Chol-TEG represents the targeting ligand having the following structure:

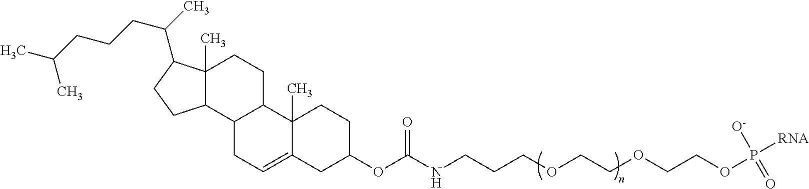

(Chol-TEG), wherein RNA comprises the remainder of the double-stranded RNAi trigger molecule, and n=2.

In some embodiments, the antisense sequence of SEQ ID NO: 5 can be annealed to the sense strand sequence of SEQ ID NO: 6, to form Duplex Pair SEQ ID NO: 5/6 (also referred to herein as AD00370). As used herein, the term “duplex pair” refers to a double stranded expression-inhibiting oligomeric compound. Additional sequences for expression-inhibiting oligomeric compounds targeting AAT are known in the art.

For the expression-inhibiting oligomeric compounds suitable for use in the methods described herein, the nucleosides, or nucleotide bases, may be linked by phosphate-containing (natural) or non-phosphate-containing (non-natural) covalent internucleoside linkages, i.e. the expression-inhibiting oligomeric compounds may have natural or non-natural oligonucleotide backbones. The expression-inhibiting oligomeric compounds may also contain a non-standard (non-phosphate) linkage between two nucleotide bases.

In some embodiments, one or more nucleotides of the expression-inhibiting oligomeric compound are modified nucleotides. In some embodiments, at least 50%, at least 60%, at least 70%, at least 80%, at least 90%, at least 95%, or 100% of the nucleotides are modified. Modified nucleotides include, but are not limited to: 2′ modifications, 2′-O-methyl nucleotide (represented herein as a lower case letter ‘n’ in a nucleotide sequence), 2′-deoxy-2′-fluoro nucleotide (represented herein as Nf, also represented herein as 2′ fluoro nucleotide), 2′-deoxy nucleotide (represented herein as dN), 2′-amino nucleotide, 2′-alkyl nucleotide, terminal 3′ to 3′ linkages, inverted deoxythymidine (represented herein as invdT), a nucleotide comprising a 5′-phosphorothioate group (represented herein as a lower case ‘s° before a nucleotide, as in sN), thiophosphate linkages, phosphorodithioate group, non-natural base comprising nucleotide; locked nucleotides, bridged nucleotides, peptide nucleic acids, 2’,3′-seco nucleotide mimic (unlocked nucleotide, represented herein as N_{U}NA), morpholino nucleotides, and abasic nucleotide. It is not necessary for all positions in a given compound to be uniformly modified. Conversely, more than one modification may be incorporated in a single expression-inhibiting oligomeric compound or even in a single nucleotide thereof. For example, a Ribose 2′ modification may be combined with modified nucleoside linkages.

The nucleobases of the expression-inhibiting oligomeric compounds may also be modified. Modified nucleobases may include other synthetic and natural nucleobases, such as 5-substituted pyrimidines, 6-azapyrimidines and N-2, N-6 and O-6 substituted purines, including 2-aminopropyladenine, 5-propynyluracil and 5-propynylcytosine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

The expression-inhibiting oligomeric compounds may also include one or more nucleotides linked by non-standard linkages or backbones (i.e. modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate group (represented herein as a lower case ‘s’ before a nucleotide, as in sN, sn, sNf, or sdN), chiral phosphorothioates, thiophosphate, phosphorodithioates, phosphotriesters, aminoalkyl-phosphotriesters, methyl and other alkyl phosphonates including 3′-alkylene phosphonates and chiral phosphonates, phosphinates, phosphoramidates including 3 ‘-amino phosphoramidate and aminoalkylphosphoramidates, thionophosphoramidates, thionoalkyl-phosphonates, thionoalkylphosphotriesters, morpholino linkages, and boranophosphates having normal 3’-5′ linkages, 2′-5′ linked analogs of these, and those having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide, sulfoxide and sulfone backbones; formacetyl and thioformacetyl backbones, methylene formacetyl and thioformacetyl backbones, alkene containing backbones, sulfamate backbones, methyleneimino and methylenehydrazino backbones, sulfonate and sulfonamide backbones, amide backbones; and others having mixed N, O, S, and CH₂ component parts.

In some embodiments, AAT expression-inhibiting oligomeric compounds may be conjugated to one or more non-nucleotide groups including, but not limited to, targeting group(s), linking group(s), delivery polymer(s), or delivery vehicle(s). The non-nucleotide group can enhance targeting, delivery or attachment of the AAT expression-inhibiting oligomeric compounds. The non-nucleotide group can be covalently linked to the 3′ and/or 5′ end of an AAT expression-inhibiting oligomeric compound. In some embodiments, an expression-inhibiting oligomeric compound contains a non-nucleotide group linked to the 3′ and/or 5′ end of the oligonucleotide. In some embodiments a non-nucleotide group is linked to the 5′ end of the sense strand of a double-stranded AAT expression-inhibiting oligomeric compound. A non-nucleotide group may be linked directly or indirectly to an expression-inhibiting oligomeric compound via a linker/linking group. In some embodiments, a non-nucleotide group is linked to the AAT expression-inhibiting oligomeric compound via a labile, cleavable, or reversible bond or linker.

In some embodiments, a non-nucleotide group enhances the pharmacokinetic or biodistribution properties of an AAT expression-inhibiting oligomeric compound or conjugate to which it is attached to improve cell- or tissue-specific distribution and cell-specific uptake of the conjugate. In some embodiments, a non-nucleotide group enhances endocytosis of the AAT expression-inhibiting oligomeric compound.

A targeting group can be monovalent, divalent, trivalent, tetravalent, or have higher valency. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an AAT expression-inhibiting oligomeric compound using a linker, such as a PEG linker or one, two, or three abasic and/or ribitol groups. In some embodiments, a targeting group comprises a galactose cluster.

In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoylgalactos-amine (see for example: Iobst, S. T. and Drickamer, K. J.B.C. 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945). Galactose derivatives have been used to target molecules to hepatocytes in vivo through their binding to the asialoglycoprotein receptor (ASGPr) expressed on the surface of hepatocytes. Binding of ASGPr ligands to the ASGPr(s) facilitates cell-specific targeting to hepatocytes and endocytosis of the molecule into hepatocytes. The galactose derivative or galactose cluster may be attached to the 3′ or 5′ end of the AAT expression-inhibiting oligomeric compound using methods known in the art.

As used herein, a galactose cluster comprises a molecule having two to four terminal galactose derivatives. A terminal galactose derivative is attached to a molecule through its C-1 carbon. In some embodiments, the galactose cluster is a galactose derivative trimer, tri-antennary galactose derivative, or tri-valent galactose derivative. In some embodiments, the galactose cluster comprises N-acetylgalactosamines (GalNAc). In some embodiments, the galactose cluster comprises a tri-valent N-acetyl-galactosamine. In some embodiments, the galactose cluster is a galactose derivative tetramer, tetra-antennary galactose derivative, or tetra-valent galactose derivative. In some embodiments, the galactose cluster comprises a tetra-valent N-acetyl-galactosamine.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al. J. Med. Chem. 1995 Vol. 39 p. 1538-1546). In some embodiments, the PEG spacer is a PEGS spacer. The branch point can be any small molecule which permits attachment of three galactose derivatives and further permits attachment of the branch point to the AAT expression-inhibiting oligomeric compound. An example of branch point group is a di-lysine or di-glutamate. Attachment of the branch point to the AAT expression-inhibiting oligomeric compound can occur through a linker or spacer. In some embodiments, the linker or spacer comprises a flexible hydrophilic spacer, such as, but not limited to: a PEG spacer. In some embodiments, a PEG spacer is a PEGS spacer (three ethylene units). In other embodiments, the PEG spacer has 1 to 20 ethylene units (PEG₁ to PEG₂₀). In some embodiments, a galactose derivative comprises an N-acetylgalactosamine (GalNAc or NAG). In some embodiments, the galactose cluster is comprised of a galactose derivative tetramer, which can be, for example, an N-acetyl-galactosamine tetramer.

In some embodiments, a linking group is conjugated to the AAT expression-inhibiting oligomeric compound. The linking group facilitates covalent linkage of the agent to a targeting group or delivery polymer or delivery vehicle. The linking group can be linked to the 3′ or the 5′ end of the AAT expression-inhibiting oligomeric compound. In some embodiments, the linking group is linked to the sense strand of a double-stranded AAT expression-inhibiting oligomeric compound. Examples of linking groups, include, but are not limited to compounds having reactive groups such a primary amines and alkynes, alkyl groups, abasic ribose, ribitol, and/or PEG groups.

A linker or linking group is a connection between two atoms that links one chemical group or segment of interest to another chemical group or segment of interest via one or more covalent bonds. A labile linkage contains a labile bond. A linkage may optionally include a spacer that increases the distance between the two joined atoms. A spacer may further add flexibility and/or length to the linkage. Spacers may include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

The AAT expression-inhibiting oligomeric compounds described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with an AAT expression-inhibiting oligomeric compounds for use in the methods disclosed herein.

In some embodiments, the AAT expression-inhibiting oligomeric compounds can be combined with lipids, nanoparticles, polymers, liposomes, micelles, polymers (such as dynamic polyconjugates or DPCs (see, e.g., WO 2000/053722, WO 2008/0022309, WO 2011/104169, and WO 2012/083185, each of which is incorporated herein by reference), or other delivery systems available in the art. The AAT expression-inhibiting oligomeric compounds can also be chemically conjugated to targeting groups, lipids (including, but not limited to cholesterol and cholesteryl derivatives), nanoparticles, polymers, liposomes, micelles, DPCs, or other delivery systems available in the art. In some embodiments, the AAT expression-inhibiting oligomeric compounds are conjugated to a delivery polymer. In some embodiments, the delivery polymer is a reversibly masked/modified amphipathic membrane active polyamine.

In some embodiments, an AAT expression-inhibiting oligomeric compound is linked to a targeting ligand that comprises an asialoglycoprotein receptor ligand. In some embodiments, an AAT expression-inhibiting oligomeric compound is linked to a targeting ligand that comprises or consists of a galactose cluster. In some embodiments, an AAT expression-inhibiting oligomeric compound is linked to a targeting ligand comprising N-acetyl-galactosamine. In some embodiments, an AAT expression-inhibiting oligomeric compound is linked to an N-acetyl-galactosamine trimer or tetramer. In some embodiments, a delivery vehicle, such as a polymer, an amphipathic polymer, a membrane active polymer, a peptide, such as a melittin or melittin-like peptide (MLP) delivery polymer, a reversibly modified polymer or peptide, or a lipid, may be used with the AAT expression-inhibiting oligomeric compounds.

Compositions Comprising AAT Expression-Inhibiting Oligomeric Compounds.

An AAT expression-inhibiting oligomeric compound can be used to inhibit expression of AAT in a cell, group of cells, or a tissue, e.g., in a subject (such as a mammal). In some embodiments, an AAT expression-inhibiting oligomeric compound is used in the formulation of a composition, i.e., a pharmaceutical composition or medicament, for administering to a subject. As used herein, a pharmaceutical composition or medicament comprises a pharmacologically effective amount of at least one AAT expression-inhibiting oligomeric compound and one or more pharmaceutically acceptable excipients. Pharmaceutically acceptable excipients (excipients) are substances other than the Active Pharmaceutical ingredient (API, therapeutic product, e.g., AAT expression-inhibiting oligomeric compound) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues or isolated organs that express or comprise the herein defined RNAi agents may be used as “pharmaceutical compositions.”

The methods disclosed herein provide for the delivery of AAT expression-inhibiting oligomeric compounds to liver cells in a mammal in vivo. Delivery of AAT expression-inhibiting oligomeric compounds may be done by any method generally known in the art, including by local administration, (e.g., direct injection, implantation, or topical administering), systemic administration, or subcutaneous, intravenous, oral, intraperitoneal, or parenteral routes, including intracranial (e.g., intraventricular, intraparenchymal and intrathecal), intramuscular, transdermal, airway (aerosol), nasal, rectal, or topical (including buccal and sublingual) administration, In certain embodiments, the compositions are administered by subcutaneous or intravenous infusion or injection.

In general, methods of administering drugs and nucleic acids for treatment of a subject are well known in the art and can be applied to administration of the AAT expression-inhibiting oligomeric compounds described herein. The AAT expression-inhibiting oligomeric compounds described herein can be administered via any suitable route in a preparation appropriately tailored to the particular route. Thus, the AAT expression-inhibiting oligomeric compounds described herein can be administered by injection, for example, intravenously, intramuscularly, intracutaneously, subcutaneously, or intraperitoneally.

In some embodiments, the methods described herein comprise combining a described AAT expression-inhibiting oligomeric compound with one or more additional therapeutics or treatments including, but not limited to: a second AAT expression-inhibiting oligomeric compound or other expression-inhibiting oligomeric compounds, a small molecule drug, an antibody, an antibody fragment, and/or a vaccine.

The described AAT expression-inhibiting oligomeric compounds and pharmaceutical compositions comprising AAT expression-inhibiting oligomeric compounds may be packaged or included in a kit, container, pack, or dispenser. The AAT expression-inhibiting oligomeric compounds and pharmaceutical compositions comprising said AAT expression-inhibiting oligomeric compounds may be packaged in pre-filled syringes or vials.

In some embodiments, pharmaceutical compositions comprising at least one AAT expression-inhibiting oligomeric compound are contemplated. These pharmaceutical compositions are useful in the inhibition of the expression of the AAT gene in a cell, a tissue, or an organism. In some embodiments, the described pharmaceutical compositions are used to treat a subject having a disease, condition, or disorder that would benefit from reduction or inhibition in AAT expression. In some embodiments, the described pharmaceutical compositions are used to treat a subject at risk of developing a disease, condition, or disorder that would benefit from reduction or inhibition in AAT expression. In some embodiments, the subject is a mammal, including, but not limited to, a human.

Cells, tissues, and non-human organisms that include at least one AAT expression-inhibiting oligomeric compound is contemplated. The cell, tissue, or non-human organism is made by delivering an AAT expression-inhibiting oligomeric compound to the cell, tissue, or non-human organism by any means available in the art. In some embodiments, the cell is a mammalian cell, including, but not limited to, a human cell. The cell, tissue, or non-human organisms are useful for research or as research tools (e.g., drug testing or diagnoses).

In some embodiments, the AAT expression-inhibiting oligomeric compounds described herein are used to treat a subject having a disease, condition, or disorder or at risk of having a disease, condition, or disorder that would benefit from reduction or inhibition in AAT expression. Treatment of a subject that would benefit from a reduction and/or inhibition of AAT expression including therapeutic and/or prophylactic treatment. Examples of diseases, conditions, or disorders of the liver caused by AATD, include, but are not limited to: injury in the liver include chronic hepatitis, cirrhosis, hepatocellular carcinoma, transaminitis, cholestasis, fibrosis, and fulminant hepatic failure. In some embodiments, the methods disclosed herein comprise administering a composition, such as a pharmaceutical composition, comprising an AAT expression-inhibiting oligomeric compound to a mammal to be treated.

In some embodiments, the methods disclosed herein comprise the administration of a therapeutically effective amount of one or more of the described AAT expression-inhibiting oligomeric compounds to a subject, thereby inhibiting expression of AAT in the subject (e.g., an amount effective to inhibit expression of AAT in the subject). In some embodiments, the methods disclosed herein comprise the administration of one or more AAT expression-inhibiting oligomeric compounds to treat a subject having a disease or disorder that would benefit from reduction or inhibition in AAT expression. In some embodiments, the methods described herein comprise administering AAT expression-inhibiting oligomeric compounds to treat or prevent at least one symptom in a subject having a disease or disorder that would benefit from reduction or inhibition in AAT. In some embodiments, the methods disclosed herein comprise the administration of a therapeutically effective amount of any one or more of the described AAT expression-inhibiting oligomeric compounds. In some embodiments, the method comprises the administration of a prophylactically effective amount of any one or more of the described AAT expression-inhibiting oligomeric compounds to a subject thereby preventing and or minimizing the severity of at least one symptom.

In some embodiments, the methods disclosed herein comprise the administration of an AAT expression-inhibiting oligomeric compound to treat or manage a clinical presentation wherein a subject in need of such treatment, prevention, or management is administered a therapeutically or prophylactically effective amount of one or more of the AAT expression-inhibiting oligomeric compounds. In some embodiments, the methods disclosed herein comprise the administration of an AAT expression-inhibiting oligomeric compound by administering an AAT expression-inhibiting oligomeric compound-containing composition as described herein. In some embodiments, the methods disclosed herein comprise administering a composition comprising one or more AAT expression-inhibiting oligomeric compounds described herein to a mammal.

In some embodiments, the methods disclosed herein further comprise the step of administering a second therapeutic or treatment to inhibit the expression of AAT. In some embodiments, the second therapeutic is another AAT expression-inhibiting oligomeric compound (e.g., an AAT expression-inhibiting oligomeric compound which targets a different sequence within the AAT gene).

In other embodiments, the methods disclosed herein further comprise the step of administering an additional therapeutic such as a small molecule drug, antibody, antibody fragment, or vaccine.

Route of Administration.

The route of administration is the path by which expression-inhibiting oligomeric compounds are brought into contact with the body. In general, methods of administering drugs and nucleic acids for treatment of a mammal are well known in the art and can be applied to administration of the compositions described herein. The expression-inhibiting oligomeric compounds disclosed herein can be administered via any suitable route in a preparation appropriately tailored to the particular route. Expression-inhibiting oligomeric compounds can be administered in various manners depending, for example, upon whether local or systemic treatment is desired and upon the area to be treated. Thus, the expression-inhibiting oligomeric compounds can be administered by injection, for example, intravenously, intramuscularly, intracutaneously, subcutaneously, or intraperitoneally. Administration can also be topical (e.g., by a transdermal patch), pulmonary, e.g., by inhalation or insufflation of powders or aerosols, including by nebulizer, intratracheal, intranasal, epidermal and transdermal, oral or parenteral. Parenteral administration would include intravenous, intraarterial, subcutaneous, intraperitoneal or intramuscular injection or infusion; subdermal, e.g., via an implanted device; or intracranial, e.g., by intraparenchymal, intrathecal or intraventricular, administration. In certain embodiments, delivery of the expression-inhibiting oligomeric compounds can be by local administration, (e.g., direct injection, implantation, or topical administering), systemic administration, or subcutaneous, intravenous, oral, intraperitoneal, or parenteral routes, including intracranial (e.g., intraventricular, intraparenchymal and intrathecal), intramuscular, transdermal, airway (aerosol), nasal, rectal, or topical (including buccal and sublingual) administration. In some embodiments, the compositions are administered by subcutaneous or intravenous infusion or injection.

In some embodiments, an expression-inhibiting oligomeric compound is administered in an unbuffered solution. In some embodiments, the unbuffered solution is saline or water. In some embodiments, an expression-inhibiting oligomeric compound is administered with a buffer solution. In some embodiments, the buffer solution comprises acetate, citrate, prolamine, carbonate, or phosphate or any combination thereof. In some embodiments, the buffer solution is phosphate buffered saline (PBS).

In some embodiments, pharmaceutical compositions may comprise at least one, and optionally two or more AAT expression-inhibiting oligomeric compounds. These pharmaceutical compositions are particularly useful in the inhibition of the expression of the AAT gene in a cell, a tissue, or an organism. The described pharmaceutical compositions can be used to treat a subject having a disease or disorder that would benefit from reduction or inhibition in AAT expression. The described pharmaceutical compositions can be used to treat a subject at risk of developing a disease or disorder that would benefit from reduction or inhibition in AAT expression. Diseases and/or disorders that would benefit from reduction or inhibition in AAT expression may be selected from the list comprising: AATD, chronic hepatitis, cirrhosis, hepatocellular carcinoma, and fulminant hepatic failure. Preferably, the subject is a mammal, most preferably a human patient.

Dosing.

In one aspect, the methods described herein comprise the administration of a pharmaceutical composition for inhibiting expression of an AAT gene comprising one or more AAT expression-inhibiting oligomeric compounds in combination with a pharmaceutical composition for reducing, removing, and/or eliminating accumulated Z-AAT globules comprising one or more autophagy enhancing agents. Administration of an AAT expression-inhibiting oligomeric compound and an autophagy enhancing agent according to the methods and uses described herein may result in a reduction of the severity, signs, symptoms, and/or markers of such diseases or disorders in a subject having a disorder that would benefit from inhibiting or reducing the expression of AAT, such as AATD. Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention.

The methods disclosed herein comprising the administration of AAT expression-inhibiting oligomeric compounds and autophagy enhancing agents can be used to treat or prevent at least one or more symptoms in a subject having a disease or disorder that would benefit from reduction or inhibition in AAT expression. In some embodiments, the subject may be administered a prophylactically effective amount of any one or more of the described expression-inhibiting oligomeric compounds and any one or more of the autophagy enhancing agents thereby preventing the at least one symptom. In some embodiments, the subject may also be administered a therapeutically effective amount of any one or more of the described expression-inhibiting oligomeric compounds and one or more of the autophagy enhancing agents thereby treating the symptom.

In some embodiments, the gene expression level and/or mRNA level of AAT in a subject to whom a described AAT expression-inhibiting oligomeric compound is administered is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject not receiving the AAT expression-inhibiting oligomeric compound. The gene expression level and/or mRNA level in the subject may be reduced in a cell, group of cells, and/or tissue of the subject. In some embodiments, the protein level of AAT in a subject to whom a described AAT expression-inhibiting oligomeric compound is administered is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject not receiving the AAT expression-inhibiting oligomeric compound. The protein level in the subject may be reduced in a cell, group of cells, tissue, blood, and/or other fluid of the subject. Reduction in gene expression, mRNA, or protein levels can be assessed by any methods known in the art. Reduction or decrease in AAT mRNA level and/or protein level are collectively referred to herein as a reduction or decrease in AAT or inhibiting or reducing the expression of AAT.

The AAT expression-inhibiting oligomeric compound dose can be: 0.0005, 0.001, 0.005, 0.01, 0.02, 0.03, 0.04, 0.05, 0.06, 0.07, 0.08, 0.09, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or 50 mg/kg. Values intermediate to the recited values are also intended to be part of this invention.

In some embodiments, the AAT expression-inhibiting oligomeric compound dose can be: 0.1 to 50, 0.25 to 50, 0.5 to 50, 0.75 to 50, 1 to 50, 1.5 to 50, 2 to 50, 2.5 to 50, 3 to 50, 3.5 to 50, 4 to 50, 4.5 to 50, 5 to 50, 7.5 to 50, 10 to 50, 15 to 50, 20 to 50, 20 to 50, 25 to 50, 25 to 50, 30 to 50, 35 to 50, 40 to 50, or 45 to 50 mg/kg.

In other embodiments, the AAT expression-inhibiting oligomeric compound dose can be: 0.1 to 45, 0.25 to 45, 0.5 to 45, 0.75 to 45, 1 to 45, 1.5 to 45, 2 to 45, 2.5 to 45, 3 to 45, 3.5 to 45, 4 to 45, 4.5 to 45, 5 to 45, 7.5 to 45, 10 to 45, 15 to 45, 20 to 45, 20 to 45, 25 to 45, 25 to 45, 30 to 45, 35 to 45, or 40 to 45 mg/kg.

In other embodiments, the AAT expression-inhibiting oligomeric compound dose can be: 0.1 to 40, 0.25 to 40, 0.5 to 40, 0.75 to 40, 1 to 40, 1.5 to 40, 2 to 40, 2.5 to 40, 3 to 40, 3.5 to 40, 4 to 40, 4.5 to 40, 5 to 40, 7.5 to 40, 10 to 40, 15 to 40, 20 to 40, 20 to 40, 25 to 40, 25 to 40, 30 to 40, or 35 to 40 mg/kg.

In other embodiments, the AAT expression-inhibiting oligomeric compound dose can be: 0.1 to 30, 0.25 to 30, 0.5 to 30, 0.75 to 30, 1 to 30, 1.5 to 30, 2 to 30, 2.5 to 30, 3 to 30, 3.5 to 30, 4 to 30, 4.5 to 30, 5 to 30, 7.5 to 30, 10 to 30, 15 to 30, 20 to 30, 20 to 30, 25 to 30 mg/kg.

In other embodiments, the AAT expression-inhibiting oligomeric compound dose can be: 0.1 to 20, 0.25 to 20, 0.5 to 20, 0.75 to 20, 1 to 20, 1.5 to 20, 2 to 20, 2.5 to 20, 3 to 20, 3.5 to 20, 4 to 20, 4.5 to 20, 5 to 20, 7.5 to 20, 10 to 20, or 15 to 20 mg/kg.

In other embodiments, the AAT expression-inhibiting oligomeric compound dose can be: 0.01 to 10, 0.05 to 10, 0.1 to 10, 0.2 to 10, 0.3 to 10, 0.4 to 10, 0.5 to 10, 1 to 10, 1.5 to 10, 2 to 10, 2.5 to 10, 3 to 10, 3.5 to 10, 4 to 10, 4.5 to 10, 5 to 10, 5.5 to 10, 6 to 10, 6.5 to 10, 7 to 10, 7.5 to 10, 8 to 10, 8.5 to 10, 9 to 10, or 9.5 to 10 mg/kg.

In other embodiments, the AAT expression-inhibiting oligomeric compound dose can be: 0.01 to 5, 0.05 to 5, 0.1 to 5, 0.2 to 5, 0.3 to 5, 0.4 to 5, 0.5 to 5, 1 to 5, 1.5 to 5, 2 to 5, 2.5 to 5, 3 to 5, 3.5 to 5, 4 to 5, or 4.5 to 5 mg/kg.

In other embodiments, the AAT expression-inhibiting oligomeric compound dose can be: 0.01 to 3, 0.05 to 3, 0.1 to 3, 0.2 to 3, 0.3 to 3, 0.4 to 3, 0.5 to 3, 1 to 3, 1.5 to 3, 2 to 3, or 2.5 to 3 mg/kg.

In other embodiments, the AAT expression-inhibiting oligomeric compound dose can be: 0.01 to 2.5, 0.05 to 2.5, 0.1 to 2.5, 0.2 to 2.5, 0.3 to 2.5, 0.4 to 2.5, 0.5 to 5, 1 to 2.5, 1.5 to 2.5, or 2 to 2.5 mg/kg.

In some embodiments, the AAT expression-inhibiting oligomeric compound dose can be 0.5 mg/kg, 1 mg/kg, 2 mg/kg, or 3 mg/kg.

In some embodiments, the AAT expression-inhibiting oligomeric compound dose can be a fixed dose. In some embodiments, the fixed dose for an AAT expression-inhibiting oligomeric compound dose approximates a dose of 0.5 mg/kg, 1 mg/kg, 2 mg/kg, or 3 mg/kg in a standard adult or child, as applicable.

Inhibition of AAT Expression.

In some embodiments, the gene expression level and/or mRNA level of AAT in a subject to whom a described AAT expression-inhibiting oligomeric compound is administered is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered the AAT expression-inhibiting oligomeric compound or to a subject not receiving an AAT expression-inhibiting oligomeric compound. The gene expression level and/or mRNA level in the subject may be reduced in a cell, group of cells, and/or tissue of the subject. In some embodiments, the protein level of AAT in a subject to whom a described AAT expression-inhibiting oligomeric compound has been administered is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject prior to being administered an AAT expression-inhibiting oligomeric compound or to a subject not receiving an AAT expression-inhibiting oligomeric compound. The protein level in the subject may be reduced in a cell, group of cells, tissue, blood, and/or other fluid of the subject. A reduction in gene expression, mRNA, or protein levels can be assessed by any methods known in the art. Reduction or decrease in AAT mRNA level and/or protein level are collectively referred to herein as a reduction or decrease in AAT or inhibiting or reducing the expression of AAT.

### Autophagy Enhancing Agents

Autophagy is an endogenous pathway to maintain cell function by targeting intracellular components such as proteins or organelles for degradation. In general, autophagy operates by way of a mechanism in which cytosolic proteins or organelles are encircled by a double-membrane vesicle called an autophagosome, which then subsequently fuses with a lysosome for degradation of the intracellular component.

The autophagy enhancing agents disclosed herein are compounds and compositions that enhance the autophagy pathways in hepatocytes leading to the enhanced clearance of Z-AAT protein globules and reduction of Z-AAT protein agglomerates retained in the ER. Various classes of known autophagy enhancing agents have been observed to have an autophagy enhancing effect on aggregate-prone proteins, such as Z-AAT, in cells or experimental animal models, which are shown in the following Table 4:

TABLE 4
Properties and information concerning certain autophagy enhancing agents.

| | | | Demonstrated |
|---|---|---|---|
| | | Reported Pathway of | Autophagy Effect on |
| | Therapeutic | Activity With Respect | Aggregate-Prone |
| Drug | Indication(s) | To Autophagy | Proteins |
| Ezetimibe | cholesterol lowering | Niemann-Pick-Type C1- | Human cell line, human |
| | | Like1, mTORC1 | primary hepatocytes, |
| | | complex | mouse small intestine |
| Statins | cholesterol lowering | Depletion of GGDP, | unknown |
| | | activation of AMPK | |
| Carbamazepine | mood stabilizer, | Inositol pathway | Rat, monkey and |
| | trigeminal neuralgia, | | human cell lines, PiZ |
| | epilepsy | | mice |
| Oxcarbazepine | treatment of partial | Inositol pathway | unknown |
| | seizures | | |
| Verapamil | angina, arrhythmias | Ca²⁺ channel blocker | Rat cell line, |
| | including | | Drosophila, zebrafish |
| | supraventricular | | |
| | tachyarrhythmias, | | |
| | hypertension | | |
| Loperamide | antidiarrheal | Ca²⁺ channel blocker | Rat cell line, |
| | | | Drosophila, zebrafish |
| Nimodipine | antihypertensive for | Ca²⁺ channel blocker | Rat cell line, |
| | reduction in brain | | Drosophila, zebrafish |
| | bleeding in patients with | | |
| | subarachnoid hemorrhage | | |
| Nitrendipine | hypertension | Ca²⁺ channel blocker | Rat cell line, |
| | | | Drosophila, zebrafish |
| Niguldipine | N/A | Ca²⁺ channel blocker | High-throughput |
| | | | screening (HTS) |
| Amiodarone | anti-arrhythmia | Ca²⁺ channel blocker | Rat cell line, |
| | | | Drosophila, zebrafish |
| Clonidine | hypertension, severe | Gᵢ protein signaling | Rat cell line, |
| | pain, ADHD | pathway via adrenergic | Drosophila, zebrafish |
| | | and imidazoline | |
| | | receptors | |
| Rilmenidine | hypertension | Gᵢ protein signaling | Rat cell line, |
| | | pathway via adrenergic | Drosophila, zebrafish |
| | | and imidazoline | |
| | | receptors | |
| Lithium | mood stabilizer, manic | Inositol pathway | Rat, monkey and |
| | depressive disorder, | | human cell lines |
| | bipolar disorder | | |
| Valproic acid | mood stabilizer | Inositol pathway | HTS, Drosophila |
| Fluphenizine | mood stabilizer, | Reduction of | C. elegans, human |
| | schizophrenia | intracellular Ca²⁺, | cells, PiZ mice |
| | | calpain inhibitor | |
| Calpastatin | N/A | Calpain inhibitors | HTS, Zebrafish |
| Pimozide | mood stabilizer, | Ca²⁺ channel blocker | HTS |
| | suppression of tics in | | |
| | patients with | | |
| | Tourette's Disorder | | |
| Fluspirilene | mood stabilizer, | Reduction of | HTS |
| | schizophrenia | intracellular Ca²⁺ | |
| Glyburide | antidiabetic, glycemic | Reduction of | PiZ mice |
| | control in patients with | intracellular Ca²⁺ | |
| | type 2 diabetes | | |
| | mellitus | | |
| Metformin | antidiabetic | Upregulation of AMPK | unknown |
| Rapamycin (sirolimus) | immunosuppressant | mTORC1 complex | Rat, monkey, and |
| and “rapalogs” | | | human cells |
| Minoxidil | hypertension | K⁺_{ATP} channel opener | Mammalian, cells, |
| | | | Drosophila, zebrafish |
| Trehalose | N/A | mTOR-independent | Mammalian cells |
| | | pathway | |
| Tyrosine kinase | anticancer | Inhibition of Akt-mTOR | unknown |
| inhibitors | | signaling | |
| Beclin 1 peptide | N/A (evaluated for | Beclin-1 complex | Mammalian cells, mice |
| | autophagy-inducing | | |
| | properties) | | |
| Expression-inhibiting | N/A | any pathway affecting | not yet demonstrated |
| oligomeric compound | | autophagy | |
| that targets the mRNA | | | |
| of an inhibitor of | | | |
| autophagy | | | |
| Bile acid derivatives, | cholestatic liver injury, | unknown | PiZ mice, humans |
| Ursodeoxycholic acid, | biliary fibrosis and | | (clinical) |
| Nor-ursodeoxycholic | AATD | | |
| acid | | | |

Treatment with autophagy enhancing agents in combination with AAT expression-inhibiting oligomeric compounds can lead to a greater effect on globule-containing hepatocytes, compared to those only treated with autophagy enhancing agents alone.

Autophagy enhancing agents are typically divided into the following two categories: (1) agents that act directly or indirectly on the mammalian target of rapamycin (mTOR) signaling pathway complex to induce autophagy, or (2) mTOR-independent mechanisms, (Chu et al., BioMed Research Intl (Article ID 459823) (2014)), mTOR is a protein kinase responsible for regulating a host of functions including cell proliferation and growth, cell survival, protein synthesis, transcription, and autophagy. Examples of drugs that are believed to act directly or indirectly through the mTOR signaling complex include, for example, rapamycin and rapamycin analogs, PI3K inhibitors, and ezetimibe. Examples of autophagy enhancing agents that act under mTOR-independent mechanisms include agents that reduce intracellular Ca²⁺ to prevent calapain-1-mediated cleavage of the autophagy gene, such as fluspirilene, or compounds that directly or indirectly impact inositol thus acting on the phosphatidylinositol signaling pathway, such as carbamazepine, valproic acid, and lithium. (Chu et al., BioMed Research Int'l (Article ID 459823) (2014)).

Non-limiting examples of autophagy enhancing agents include the following:

Ezetimibe.

Ezetimibe is known as a potent inhibitor of cholesterol absorption in the small intestine. (Garcia-Calvo, PNAS 102(23): 8132-8137 (2005)). Ezetimibe is an active ingredient included the FDA-approved branded drug Zetia®, which is indicated for lowering plasma cholesterol levels. Zetia® (ezetimibe) is a 10 mg tablet that is to be administered once daily.

With respect to autophagy, ezetimibe and related compounds are believed to activate the autophagy mechanism by inhibiting the cholesterol efflux Niemann-Pick-type C1 like 1 (NPC1L1), which in turn reduces recruitment of mTOR to the lysosome, inhibiting mTORC1 activity. (Yamamura, 2014, Hepatology 59: 1591-1599).

Carbamazepine.

Carbamazepine is an active ingredient included in several FDA-approved branded drug products indicated for the treatment of conditions associated with epilepsy and neuropathic pain. These include Carbatrol® (100; 200 and 300 mg oral extended-release capsules indicated for use as an anticonvulsant); Equetro® (100, 200 and 300 mg oral extended-release capsules indicated for use as a mood stabilizer for the treatment of acute manic or mixed episodes associated with bipolar I disorder); Tegretol® (100 and 200 mg oral tablets and 100 mg/5 mL oral suspension indicated for use as an anticonvulsant and the treatment of pain associated with true trigeminal neuralgia); and Tegretol XR® (100, 200, and 400 mg oral extended-release tablets indicated for use as an anticonvulsant and the treatment of pain associated with true trigeminal neuralgia). The recommended FDA-approved daily doses for carbamazepine range from 10 to 20 mg/kg/day administered twice daily in small children, up to doses of 1600 mg daily in adults in rare instances.

Hidvegi et al. reported that carbamazepine was able to mediate a reduction in Z-AAT globules in hepatocytes and ameliorated hepatic fibrosis in the PiZ mouse model of AATD. (Hidvegi et al. 2010, Science 329: 229-232). Although the exact mechanism by which carbamazepine and related compounds act to reduce hepatic load of Z-AAT protein in hepatocytes is not understood, its mood-stabilizing effects are similar to the drugs lithium and valproic acid and it may operate under a similar mechanism. The mechanism is believed to involve the inhibition of inositol monophosphatase (IMPase), leading to reduced intracellular inositol levels, thereby negatively regulating autophagy. (Chu et al., BioMed Research Intl (Article ID 459823) (2014) at 4). Additionally, related compounds and derivatives of carbamazepine, such as oxcarbazepine and imipramine, may also have an autophagy enhancing effect in hepatocytes.

Fluphenazine.

Fluphenazine is a trifluoromethyl phenothiazine derivative that acts as an antipsychotic medicine, and is indicated for use in the management of patients with schizophrenia. Fluphenazine can be administered in various salt forms, including fluphenazine hydrochloride, fluphenazine decanoate, and fluphenazine enanthate. Fluphenazine hydrochloride was first marketed over 50 years ago under the brand names Permitil® and Prolixin®. Prolixin® Decanoate (fluphenazine decanoate) and Prolixin® Enanthate (fluphenazine enanthate) were also first marketed in the United States several decades ago. At least fluphenazine hydrochloride (2.5 mg/5 mL elixir; 5 mg/mL concentrate; 2.5 mg/mL injection; and 1 mg, 2.5 mg, 5 mg, and 10 mg oral tablets) and fluphenazine decanoate (25 mg/mL injection) are currently approved in the United States.

The recommended FDA-approved dosage for fluphenazine hydrochloride is typically 2.5 to 10 mg daily, to be divided and given at 6 or 8 hour intervals. Up to 40 mg daily may be necessary in severe patients. When symptoms are controlled, doses as low as 1 mg daily may be used for maintenance therapy.

The recommended FDA-approved dosage for long acting fluphenazine decanoate is generally 12.5 to 25 mg/mL as a starting dose, going up to 100 mg/mL in severe cases. Fluphenazine decanoate typically has an onset of action after 24 to 72 hours after administration, and a single 25 mg dose can be effective in controlling schizophrenic behavior for several weeks.

Li et al. reported that fluphenazine was able to mediate a reduction in Z-AAT globules in hepatocytes and ameliorated hepatic fibrosis in the PiZ mouse model of AATD (Li et al, 2014, PLOS ONE 9:e87260). Although the exact mechanism by which fluphenazine and related compounds act to reduce hepatic load of Z-AAT protein in hepatocytes is not fully understood, fluphenazine and other phenothiazine compounds may activate autophagy by modulating cellular calcium.

Pimozide.

Pimozide is an orally active antipsychotic agent of the diphenyl-butylpiperidine series having the ability to blockade dopaminergic receptors and is indicated for the suppression of motor and phonic tics in patients with Tourette's Disorder. Pimozide can be administered in various salt forms, including fluphenazine hydrochloride, fluphenazine decanoate, and fluphenazine enanthate. Pimozide was the active ingredient in the branded product Orap® (1 mg and 2 mg oral tablets). The recommended FDA-approved dosage for pimozide is as low as 0.05 mg/kg daily for children, up to 10 mg/kg daily for adults. Doses greater than 0.2 mg/kg daily or 10 mg daily are not recommended.

Pimozide and related compounds may activate autophagy by modulating cellular calcium.

Fluspirilene.

Fluspirilene is a diphenylbutylpiperidine antipsychotic drug that may be used for the treatment of schizophrenia, and was developed and previously marketed by Janssen Pharmaceutics. Fluspirilene was marketed worldwide under the brand names Imap® and

Redeptin®, and was available in 2 mg/mL and 10 mg/mL formulations.

Fluspirilene and related compounds may activate autophagy by modulating cellular calcium.

Glyburide.

Glyburide is an oral blood-glucose-lowering drug of the sulfonylurea class, and is indicated as an adjunct therapy to improve glycemic control in adults with type 2 diabetes mellitus. Micronized glyburide is the active ingredient in the branded drug product Glynase® PresTab® (1.5, 3, and 6 mg oral tablets), and in the branded drub product Diaβeta® (1.25, 2.5, and 5 mg oral tablets). Although there is no fixed dosage regimen for Glynase® PresTab®, the suggested starting does is 1.5 to 3 mg administered daily. Daily doses of more than 12 mg of Glynase® PresTab® are not recommended. Similarly, although there is no fixed dosage regimen for Diaβeta®, the suggested starting dose is 2.5 to 5 mg daily, with a usual maintenance dose of 1.25 to 20 mg daily. Daily doses of more than 20 mg of Diaβeta® are not recommended.

Glyburide and related compounds may activate autophagy by modulating cellular calcium.

Clonidine.

Clonidine is an imidazoline derivative centrally-acting alpha-agonist hypertensive agent, known to stimulate alpha-adrenoreceptors in the brain stem resulting in a decrease in heart rate and blood pressure. The alpha₂-adrenergic agonist properties of clonidine have also been shown to treat attention deficit hyperactivity disorder (ADHD). Clonidine is also known as a centrally-acting analgesic and is indicated for severe pain treatment.

Clonidine hydrochloride is the active ingredient in several FDA-approved products for the treatment of hypertension, including the branded drug products Catapres® (0.1, 0.2, and 0.3 mg oral tablets for hypertension), Catapres-TTS®-1, Catapres-TTS®-2, and Catapres-TTS®-3 (7-day transdermal patch system releasing 0.1, 0.2, and 0.3 mg per 24 hours). Clonidine hydrochloride is also the active ingredient in the FDA-approved product Kapvay® (0.1 mg and 0.2 mg extended-release tablets), treatment of ADHD. For severe pain, clonidine hydrochloride is the active ingredient in the FDA-approved products Duraclon® (1 mg/10 mL and 5 mg/10 mL for continuous epidural infusion).

The initial dose of Catapres® is 0.1 mg twice daily, with therapeutic doses commonly ranging from 0.2 mg to 0.6 mg per day in divided doses. Doses of Catapres® as high as 2.4 mg daily have been shown effective, but are rarely employed. The recommended starting dose of Kapvay® is 0.1 mg daily, which may be increased to 0.4 mg daily. The recommended starting dose of Duraclon® is 30 μg/hr, which may be titrated, and is to be diluted in 0.9% sodium chloride for injection, to a final concentration of no more than 100 μg/mL.

Clonidine and related compounds may activate autophagy by modulating cAMP levels. Additionally, related compounds and derivatives of clonidine, such as dexmedetomidine, guanfacine, xylazine, and xylometazoline, may also have an autophagy enhancing effect in hepatocytes.

Verapamil.

Verapamil is a calcium ion antagonist or slow-channel blocker that inhibits calcium ion influx. Verapamil hydrochloride has been approved for several indications, including angina, arrhythmias, and hypertension.

Verapamil hydrochloride is the active ingredient in several drug products that have been FDA-approved, including the branded drug product Calan® (40 mg, 80 mg, and 120 mg oral tablets; and 5-mg (2 ml) ampules, 5-mg (2 ml) and 10-mg (4 ml) syringes, and 5-mg (2 ml) and 10-mg (4 ml) vials, for intravenous administration), Calan® SR (120 mg, 180 mg, and 240 mg sustained-release caplets) and Covera-HS® (180 mg and 240 mg extended-release tablets). Calan® oral tablets can be initially administered at 40 mg doses three times per day, but doses can be up to 480 mg/daily in divided doses. Calan® SR oral tablets can be initially administered at 180 mg daily, but doses can be titrated upwards to 240 mg every 12 hours (480 mg total). For intravenous administration of Calan®, the recommended initial dose for adults is 5-10 mg as an intravenous bolus, with 10 mg repeated doses 30 minutes after the initial dose until the response is adequate, and doses as low as 0.1 mg/kg may be effective in children. Covera-HS® extended-release oral tablets can be initially administered at 180 mg daily, but doses can be titrated upwards to 480 mg every evening, and clinical trials tested doses of up to 540 mg administered at bedtime.

Verapamil hydrochloride is also the active ingredient in the branded drug product Isoptin® (40 mg, 80 mg, and 120 mg oral tablets; and 2.5 mg/mL for intravenous administration).

Verapamil hydrochloride is also the active ingredient in Verelan® (120, 180, 240, and 360 mg sustained-release pellet filled capsules) and Verelan® PM (100, 200, and 300 mg extended-release capsules). Verelan® can be administered up to 480 mg daily, and Verelan® PM can be administered up to 400 mg at bedtime.

Verapamil and related compounds may activate autophagy by modulating cellular calcium.

Loperamide.

Loperamide hydrochloride is a synthetic oral antidiarrheal. Loperamide hydrochloride is the active ingredient in the braded drug product Imodium® (2 mg capsules), Imodium® A-D (2 mg caplets and 1 mg/7.5 mL liquid), Imodium® A-D EZ Chews (2 mg chewable tablets), and Imodium® A-D for use in Children (1 mg/7.5 mL liquid). The recommended initial dose of Imodium® in adults is 4 mg, followed by 2 mg after each unformed stool, with a daily dose not exceeding 16 mg. For Imodium® A-D, no more than 4 caplets (8 mg) or 60 mL (8 mg) may be administered every 24 hours in adults. Imodium® A-D EZ Chews are to be administered no more than 4 tablets (8 mg) every 24 hours in adults.

Loperamide and related compounds may activate autophagy by modulating cellular calcium.

Nimodipine.

Nimodipine is a calcium channel blocker that has been approved for the improvement of neurological outcome by reducing the incidence and severity of ischemic deficits in patients with subarachnoid hemorrhage from ruptured intracranial berry aneurysms.

Nimodipine is the active ingredient in the branded drug product Nimotop® (30 mg oral capsules). The recommended dose of Nimotop® oral capsules is 60 mg dosed every four hours for 21 consecutive days (or within 96 hours of the hemorrhage ceasing). Nimodipine is also the active ingredient in the branded drug product Nymalize® (60 mg/20 mL oral solution), for which the recommended dose is 20 mL (60 mg) dosed every four hours for 21 consecutive days (or within 96 hours of the hemorrhage ceasing).

Nimodipine and related compounds may activate autophagy by modulating cellular calcium.

Nitrendipine.

Nitrendipine is a calcium channel blocker with marked vasodilatory action that and is recognized as an effective antihypertensive agent. Nitrendipine has been approved in various parts of the world for hypertension and has been known to reduce the cardiotoxicity of cocaine, and has been marketed as 10 mg and 20 mg tablets, with a recommended daily dose not exceeding 40 mg daily.

Nitrendipine and related compounds may activate autophagy by modulating cellular calcium.

Amiodarone.

Amiodarone is an anti-arrhythmia drug. Amiodarone hydrochloride is the active ingredient in the FDA-approved drugs Cordarone® (200 mg tablets) and Nextarone® (150 mg/100 mL and 360 mg/200 mL premixed injection for intravenous use), which are indicated for the treatment of life-threatening recurrent ventricular arrhythmias. Cordarone® is recommended to be administered at 800 to 1,600 mg daily as a loading dose for up to three weeks, with 600 to 800 mg daily dose at approximately one month, and a 400 mg daily dose as a usual maintenance dose. Nextarone® is recommended to be administered at approximately 1,000 mg for the first 24 hours, and mean daily doses over 2,100 mg were associated with an increased risk of hypertension in clinical studies.

Amiodarone and related compounds may activate autophagy by inhibiting mTORC1 signaling and/or through an mTOR-independent pathway by modulating cellular calcium.

Lithium.

Lithium is known to alter sodium transport in nerve and muscle cells and effect a shift toward intraneuronal metabolism of catecholamines. Lithium has been approved for the treatment of manic episodes of manic depression and bipolar disorder. Lithium carbonate is the active ingredient in several drug products, including Eskalith® (lithium carbonate 300 mg oral capsules), Eskalith CR® (lithium carbonate 450 mg controlled-release tablets), Lithobid® (300 mg extended-release tablets), and Lithonate® (300 mg oral capsules and 300 mg/5 mL oral syrup).

For Eskalith® and Eskalith CR®, most patients are reported to be stabile on 900 mg daily doses of lithium, but optimal patient response has been reported in doses of 1800 mg daily. The optimal recommended dose for Lithobid® is 1800 mg daily for acute mania, administered as 900 mg in the morning and 900 mg in the evening, and 1200 mg daily for maintenance therapy, administered as 600 mg in the morning and 600 mg in the evening. For lithium carbonate in the 300 mg/5 mL syrup formulation, optimal patient response for acute mania can usually be established with 10 mL, administered 3 times daily.

Lithium is believed to act by way of inhibition of inositol monophosphatase, leading to decreased myo-inositol-1,4,5-triphosphate OP3)

Rapamycin.

Rapamycin, also known as sirolimus, is a macrolide produced by bacterium that has immunosuppressant properties in humans. Rapamycin is the active ingredient in the FDA approved product Rapamune® (0.5 mg, 1 mg, and 2 mg oral tablets, and 60 mg/60 mL solution), which is indicated for the prophylaxis of organ rejection in patients receiving renal transplants. Depending on the patients' condition, the recommended initial starting dose is anywhere from as low as 2 mg daily, and a total maximum daily dose should not exceed 40 mg daily.

With respect to autophagy, rapamycin is believed to act by way of inhibition of mammalian target of rapamycin (mTOR), a negative regulator of autophagy. Additionally, related compounds and derivatives of rapamycin, such as temsirolimus, everolimus, deforolimus, and ATP-competitive mTOR kinase inhibitors, may also have an autophagy enhancing effect in hepatocytes.

Minoxidil.

Minoxidil is an antihypertensive peripheral vasodilator. Minoxidil is the active ingredient in the drug product Loniten® (2.5 and 10 mg tablets), which is indicated for hypertension. The recommended initial dose for Loniten® is 5 mg daily, which may be increased to 40 mg daily given in single or divided doses, with the maximum recommended daily dose of 100 mg.

Minoxidil and related compounds may activate autophagy by modulating cellular calcium.

Beclin1 peptide.

With respect to autophagy, beclin1 peptide is believed to act by way of interaction with GAPR-1 (aka GLIPR2), a negative regulator of autophagy.

Bile Acid Derivatives, Ursodeoxycholic Acid/Nor-Ursodeoxycholic Acid.

Other compounds that mimic the effect of activation or upregulation of autophagy may also be useful in combination with expression-inhibiting oligomeric compounds in the treatment of AATD and conditions, manifestations, and diseases caused by AATD. For example, ursodeoxycholic acid (UDCA) and other bile acid derivatives, including bile salts such as taurocholate and glycocholate are increasingly used for the treatment of cholestatic liver diseases. UDCA has been shown to improve clinical status and liver test results in some children with liver disease associated with AATD. (Lykavieris et al. 2008 Journal of Pediatric Gastroenterology and Nutrition 47:623-629). Experimental evidence suggests three major mechanisms of action for bile acids: (1) protection of cholangiocytes against cytotoxicity of hydrophobic bile acids, resulting from modulation of the composition of mixed phospholipid-rich micelles, reduction of bile acid cytotoxicity of bile and, possibly, decrease of the concentration of hydrophobic bile acids in the cholangiocytes; (2) stimulation of hepatobiliary secretion, putatively via Ca(2+)- and protein kinase C-alpha-dependent mechanisms and/or activation of p38 (MAPK) and extracellular signal-regulated kinases (Erk) resulting in insertion of transporter molecules (e.g., bile salt export pump, BSEP, and conjugate export pump, MRP2) into the canalicular membrane of the hepatocyte and, possibly, activation of inserted carriers; (3) protection of hepatocytes against bile acid-induced apoptosis, involving inhibition of mitochondrial membrane permeability transition (MMPT), and possibly, stimulation of a survival pathway. (Paumgartner and Beuers, 2002 Hepatology 36:525-531). A recent study in the PiZ mouse model of AATD demonstrated that the modified bile acid nor-ursodeoxycholic acid (nor-UDCA) reduced apoptotic signaling and reduced accumulation of mutant Z-AAT in hepatocytes; and these effects from nor-UDCA were associated with an increase in hepatic autophagy. (Tang et al., 2016 American Journal of Physiology—Gastrointestinal and Liver Physiology 311:G156-G165). Any agent which improves hepatocyte health as measured by, for example, ALT, AST, and GGT, such as the bile acid derivative disclosed herein, could allow for hepatocytes to more readily clear Z-AAT polymers and improve longer term outcomes in AATD compared to administration with solely AAT expression-inhibiting oligonucleotide compounds. While not intending to be bound by any theory, it is believed that bile acid derivatives may operate at least in part through the mechanism of autophagy.

The autophagy enhancing agents expressly listed herein and identified in Table 1 are merely exemplary and are not intended to be limiting to the scope of the application. Any suitable autophagy enhancing agent capable of reducing or eliminating the Z-AAT globules beyond what can be achieved through monotherapy treatment of an AAT expression-inhibiting oligomeric compound may be used. Further, various derivative compounds and analogs to the compounds expressly described herein have similar and related properties, which may allow them to act by the same or similar mechanisms with respect to autophagy or other pathways that lead to benefits in the treatment of AATD. Such compounds are envisioned to fall within the scope of the invention.

Compositions Comprising Autophagy Enhancing Agents.

The autophagy enhancing agents disclosed herein can be provided in compositions comprising a pharmaceutically acceptable carrier or pharmaceutically acceptable excipient (including, e.g., vehicles, carriers, and/or diluents). Excipients may include, but are not limited to: absorption enhancers, anti-adherents, anti-foaming agents, anti-oxidants, binders, buffering agents, carriers, coating agents, colors, delivery enhancers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents. A pharmaceutically acceptable excipient may or may not be an inert substance.

The pharmaceutical compositions can contain other additional components commonly found in pharmaceutical compositions. The pharmaceutically-active materials may include, but are not limited to: anti-pruritics, astringents, local anesthetics, or anti-inflammatory agents (e.g., antihistamine, diphenhydramine, etc.). The autophagy enhancing agents may be in the form of any suitable dosage form known in the art for the particular autophagy agent. For example, the autophagy enhancing agents disclosed herein may be administered orally in capsule or tablet form, or any other suitable unit dosage form, in association with the administration of the AAT expression-inhibiting oligomeric compound. Of course, other types of administration of both medicaments, as suitable for the specific autophagy agent selected, are contemplated, such as by nasal spray, by a buccal or sublingual administration dosage form, transdermally, parenterally, by suppository, by sustained release dosage form, etc. Any form of administration will work so long as the proper dosages are delivered.

In some embodiments, the pharmaceutical compositions comprising the autophagy enhancing agent may further comprise at least one, and optionally two or more, AAT expression-inhibiting oligomeric compounds. In some embodiments, the pharmaceutical compositions comprising the autophagy enhancing agent is separate from the pharmaceutical composition comprising the AAT expression-inhibiting oligomeric compound. The described pharmaceutical compositions can be used to treat a subject having a disease or disorder that would benefit from reduction or inhibition in AAT expression. The described pharmaceutical compositions can be used to treat a subject at risk of developing a disease or disorder that would benefit from reduction or inhibition in AAT expression and Z-AAT globule formation. Diseases and/or disorders that would benefit from such reduction or inhibition may be selected from the list comprising: AATD, chronic hepatitis, cirrhosis, hepatocellular carcinoma, and fulminant hepatic failure. Preferably, the subject is a mammal, most preferably a human patient.

Routes of Administration.

The autophagy enhancing agents disclosed herein can be administered via any suitable route in a preparation appropriately tailored to the particular route. Autophagy enhancing agents can be administered in various manners depending, for example, upon the properties of the autophagy agent compounds itself, whether local or systemic treatment is desired, and the area to be treated. Thus, the autophagy enhancing agents can be administered by injection, for example, intravenously, intramuscularly, intracutaneously, subcutaneously, or intraperitoneally. Administration can also be oral (e.g., by tablet, capsule, elixir, oral suspension, oral solution), topical (e.g., by a transdermal patch), pulmonary, e.g., by inhalation or insufflation of powders or aerosols, including by nebulizer, intratracheal, intranasal, epidermal and transdermal, or parenteral. Parenteral administration would include intravenous, intraarterial, subcutaneous, intraperitoneal or intramuscular injection or infusion; subdermal, e.g., via an implanted device; or intracranial, e.g., by intraparenchymal, intrathecal or intraventricular, administration. In certain other embodiments, delivery of the autophagy enhancing agent can be by local administration, (e.g., direct injection, implantation, or topical administering), systemic administration, or subcutaneous, intravenous, oral, intraperitoneal, or parenteral routes, including intracranial (e.g., intraventricular, intraparenchymal and intrathecal), intramuscular, transdermal, airway (aerosol), nasal, rectal, or topical (including buccal and sublingual) administration. In some embodiments, particularly in non-human mammals, the autophagy enhancing agent may be administered by oral gavage or under the skin. In some embodiments, the autophagy enhancing agent is included in drinking water, or mixed or formulated with food. In some embodiments, the autophagy enhancing agents are administered by subcutaneous or intravenous infusion or injection. In some embodiments, the autophagy enhancing agents are administered orally as tablets, capsules, liquids, chewable tablets, or other orally administered dosage forms.

Treatment and Prevention.

The described autophagy enhancing agents and methods can be used, in combination with AAT expression-inhibiting oligomeric compounds, to treat or prevent at least one or more symptoms in a subject having a disease or disorder that would benefit from reduction or elimination of Z-AAT globules in hepatocytes. In some embodiments, the subject is administered a therapeutically effective amount of any one or more of the described autophagy enhancing agents, in combination with one or more AAT expression-inhibiting oligomeric compounds, thereby treating the symptom. In other embodiments, the subject may be administered a prophylactically effective amount of any one or more of the described autophagy enhancing agents, in combination with any one or more of the AAT expression-inhibiting oligomeric compounds, thereby preventing at least one symptom.

In some embodiments, the reduction of Z-AAT globules in a subject to whom a described AAT expression-inhibiting oligomeric compound and an autophagy enhancing agent is administered is reduced by at least about 5%, 10%, 15%, 20%, 25%, 30%, 35%, 40%, 45%, 50%, 55%, 60%, 65%, 70%, 75%, 80%, 85%, 90%, 95%, or 98% relative to the subject not receiving the AAT expression-inhibiting oligomeric compound and/or the autophagy enhancing agent. The Z-AAT protein globules levels in the subject may be reduced in a cell, group of cells, tissue, blood, and/or other fluid of the subject. Reduction in gene expression, mRNA, protein levels, or Z-AAT globules can be assessed by any methods known in the art.

The above provided embodiments and items are now illustrated with the following, non-limiting examples.

### EXAMPLES

### Example 1. Liver mRNA Analysis

Compositions comprising double-stranded AAT expression-inhibiting oligomeric compounds were administered to PiZ mice. PiZ mice harbor the human PiZ AAT mutant allele and model human AATD (Carlson et al. Journal of Clinical Investigation 1989). Each mouse received a single intravenous (IV) dose of a composition comprising 6 mg/kg of Duplex Pair SEQ ID NO: 5/6 (AAT RNAi agent) with 6 mg/kg of melittin-like peptide (MLP) delivery polymer. Liver hAAT mRNA production was measured at days 3 and 10. Reduced mRNA levels correlated with decreased serum hAAT protein levels, except that mRNA reduction preceded protein reduction by a few days. The level of liver hAAT mRNA production was measured at day 3 and day 10 following a single dose of AAT RNAi agent with MLP delivery polymer in PiZ mice. A sustained decrease in liver hAAT mRNA levels was observed that correlated with the decrease observed in serum hAAT protein levels.

TABLE 5
Serum hAAT protein levels in PiZ mice following administration of
6 mg/kg of Duplex Pair SEQ ID NO: 5/6 (AAT RNAi agent) or
Luciferase siRNA control with 6 mg/kg of MLP delivery polymer.
Serum hAAT levels were normalized to day 1 and saline control.

| | Serum hAAT normalized to day 1 | | | |
|---|---|---|---|---|
| Treatment | day −2 | day 1 | day 3 | day 10 |
| Saline | 0.938 ± 0.168 | 1.00 | 1.077 ± 0.127 | — |
| Luc siRNA | 0.766 ± 0.219 | 1.00 | 1.110 ± 0.147 | — |
| control | | | | |
| AAT RNAi | 1.111 ± 0.605 | 1.00 | 0.326 ± 0.021 | — |
| agent (SEQ ID | | | | |
| NO: 5/6) | | | | |
| AAT RNAi | 0.483 ± 0.060 | 1.00 | 0.274 ± 0.072 | 0.105 ± 0.033 |
| agent (SEQ ID | | | | |
| NO: 5/6) | | | | |

TABLE 6
Liver hAAT mRNA levels in PiZ mice following administration of
6 mg/kg Duplex Pair SEQ ID NO: 5/6 (AAT RNAi agent) or Luciferase
siRNA control with 6 mg/kg MLP delivery polymer. AAT mRNA level is
expressed relative to mouse β-actin mRNA level.

hAAT mRNA level

| Treatment | day 3 | day 10 |
|---|---|---|
| Saline | 1.00 ± 0.15 | — |
| siLuc siRNA control | 1.06 ± 0.20 | — |
| AAT RNAi agent | 0.025 ± 0.02 | 0.055 ± 0.02 |
| SEQ ID NO: 5/6 | | |

### Example 2. In Vivo Dose Response for AAT Expression-Inhibiting Oligomeric Compounds (Duplex Pair SEQ ID NO: 5/6)

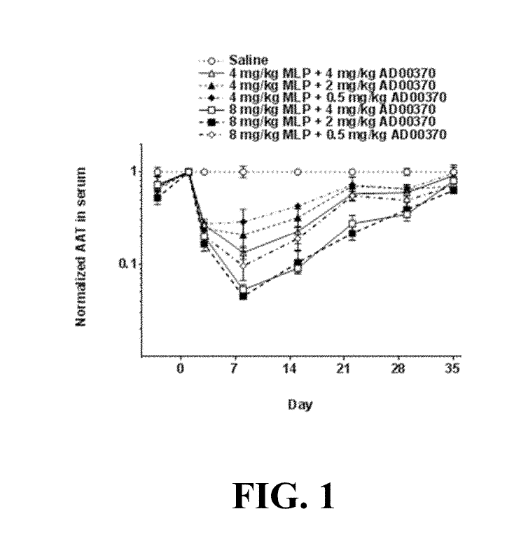

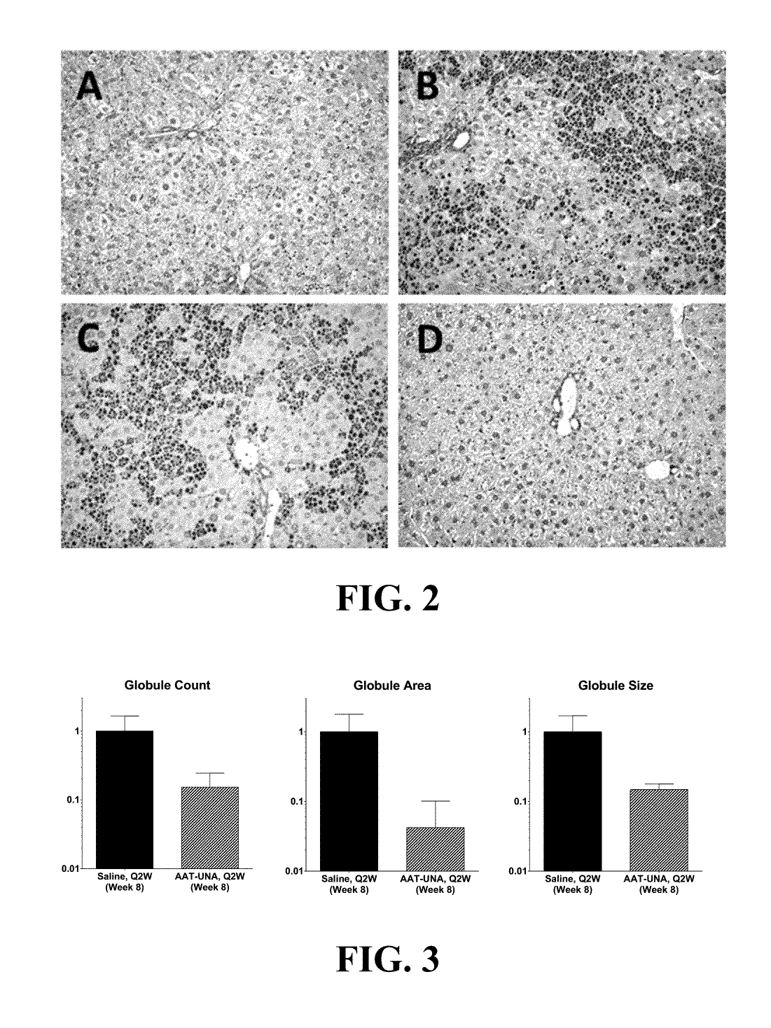

Various amounts of double-stranded AAT expression-inhibiting oligomeric compounds were administered to PiZ mice. Each mouse received a single intravenous (IV) dose of Duplex Pair SEQ ID NO: 5/6 (AAT RNAi agent) with either 4 or 8 mg/kg of MLP delivery polymer. Human AAT protein levels in serum were monitored for 35 days. The level of hAAT knockdown was largely dose dependent, in relation to both the dose of AAT RNAi agent and dose of MLP delivery polymer (See FIG. 1).

TABLE 7
Levels of serum hAAT in PiZ mice normalized to Day 1 and saline control group

| mg/kg Duplex | mg/kg MLP | | | | | | | | |
|---|---|---|---|---|---|---|---|---|---|
| Pair SEQ ID | delivery | Serum hAAT normalized to Day 1 and control | | | | | | | |
| NO: 5/6 | polymer | Day −3 | Day 1 | Day 3 | Day 8 | Day 15 | Day 22 | Day 29 | Day 35 |
| Saline control | | 1.00 ± 0.08 | 1.00 | 1.00 ± 0.02 | 1.00 ± 0.11 | 1.00 ± 0.04 | 1.00 ± 0.02 | 1.00 ± 0.06 | 1.00 ± 0.09 |
| 4 | 8 | 0.73 ± 0.13 | 1.00 | 0.20 ± 0.04 | 0.05 ± 0.01 | 0.09 ± 0.01 | 0.28 ± 0.05 | 0.35 ± 0.05 | 0.81 ± 0.12 |
| 2 | 8 | 0.53 ± 0.08 | 1.00 | 0.17 ± 0.03 | 0.05 ± 0.00 | 0.10 ± 0.03 | 0.22 ± 0.03 | 0.40 ± 0.06 | 0.63 ± 0.04 |
| 0.5 | 8 | 0.71 ± 0.03 | 1.00 | 0.20 ± 0.02 | 0.10 ± 0.03 | 0.19 ± 0.05 | 0.55 ± 0.05 | 0.49 ± 0.09 | 0.71 ± 0.09 |
| 4 | 4 | 0.70 ± 0.17 | 1.00 | 0.27 ± 0.03 | 0.13 ± 0.02 | 0.23 ± 0.07 | 0.58 ± 0.09 | 0.60 ± 0.08 | 0.92 ± 0.22 |
| 2 | 4 | 0.67 ± 0.02 | 1.00 | 0.25 ± 0.03 | 0.21 ± 0.07 | 0.32 ± 0.06 | 0.71 ± 0.14 | 0.66 ± 0.06 | 0.70 ± 0.09 |
| 0.5 | 4 | 0.64 ± 0.10 | 1.00 | 0.27 ± 0.02 | 0.29 ± 0.09 | 0.43 ± 0.01 | 0.73 ± 0.03 | 0.66 ± 0.05 | 0.97 ± 0.02 |

### Example 3. In Vivo Dose Response for AAT Expression-Inhibiting Oligomeric Compounds

Various amounts of AAT expression-inhibiting oligomeric compounds were administered to PiZ mice. Each mouse received a single intravenous (IV) dose of Duplex Pair SEQ ID NO: 5/6 (AAT RNAi agent) with either 2, 4 or 8 mg/kg of MLP delivery polymer. Human AAT protein levels in serum were monitored for 36 days. Increasing dose of AAT RNAi agent generally led to increased level and duration of knockdown for each level of MLP delivery polymer excipient used.

TABLE 8
Serum hAAT protein levels in PiZ mice following administration of varying doses of Duplex Pair SEQ ID NO: 5/6 (AAT
RNAi agent) with varying doses of MLP delivery polymer. AAT levels were normalized to day 1 and saline control.

| mg/kg Duplex | | | | | | | | |
|---|---|---|---|---|---|---|---|---|
| Pair SEQ ID | | Normalized serum hAAT levels | | | | | | |
| NO: 5/6 | mg/kg MLP | day −7 | day 1 | day 8 | day 15 | day 20 | day 29 | day 36 |
| Saline | | 1.00 ± 0.21 | 1.00 | 1.00 ± 0.14 | 1.00 ± 0.16 | 1.00 ± 0.12 | 1.00 ± 0.16 | 1.00 ± 0.13 |
| 2 | 2 | 0.91 ± 0.11 | 1.00 | 0.32 ± 0.24 | 0.88 ± 0.13 | 0.89 ± 0.18 | 1.01 ± 0.23 | 1.02 ± 0.15 |
| 4 | 2 | 1.27 ± 0.07 | 1.00 | 0.08 ± 0.03 | 0.68 ± 0.13 | 0.90 ± 0.15 | 1.07 ± 0.07 | 1.01 ± 0.08 |
| 8 | 2 | 0.70 ± 0.15 | 1.00 | 0.09 ± 0.05 | 0.59 ± 0.16 | 0.74 ± 0.10 | 0.87 ± 0.20 | 0.74 ± 0.08 |
| 2 | 4 | 0.90 ± 0.15 | 1.00 | 0.07 ± 0.04 | 0.50 ± 0.19 | 0.67 ± 0.12 | 0.89 ± 0.06 | 0.94 ± 0.17 |
| 4 | 4 | 0.68 ± 0.07 | 1.00 | 0.03 ± 0.01 | 0.23 ± 0.04 | 0.32 ± 0.05 | 0.66 ± 0.10 | 0.83 ± 0.06 |
| 8 | 4 | 0.70 ± 0.24 | 1.00 | 0.04 ± 0.02 | 0.27 ± 0.05 | 0.35 ± 0.05 | 0.80 ± 0.20 | 1.00 ± 0.20 |
| 8 | 8 | 0.89 ± 0.54 | 1.00 | 0.02 ± 0.00 | 0.13 ± 0.06 | 0.16 ± 0.04 | 0.43 ± 0.08 | 0.78 ± 0.23 |

### Example 4. Liver Histology in PiZ-Transgenic Mice Treated with AAT Expression-Inhibiting Oligomeric Compounds

To further evaluate efficacy of hAAT knockdown in the liver, histological changes were assessed in liver samples from male PiZ mice following administration of Duplex Pair SEQ ID NO: 5/6 (AAT RNAi agent) with MLP delivery polymer. AAT RNAi agent was administered to PiZ mice. Each mouse received a biweekly administration of an intravenous (IV) dose of 8 mg/kg AAT RNAi agent with 8 mg/kg of MLP delivery polymer for 8 weeks. Mice were bled weekly to monitor hAAT levels in serum and were sacrificed on day 57 after administration of AAT RNAi agent with MLP delivery polymer. Liver samples were harvested and fixed in 10% neutral-buffered formalin and embedded in paraffin. Inflammatory infiltration was assessed by H&E staining. The PiZ mice injected biweekly with 8 mg/kg AAT RNAi agent with 8 mg/kg of MLP delivery polymer for 8 weeks had normal morphology, no detectably inflammatory infiltrate and very rare, small Z-hAAT globules. PiZ mice injected biweekly with saline had significant globule accumulation as well as inflammatory infiltration around damaged or dead hepatocytes. Aggregation of Z-hAAT was visualized by performing diastase-resistant periodic acid Schiff (PAS-D) staining on liver sections. Diastase digestion of glycogen prior to performing a PAS stain allows positive staining of Z-AAT protein accumulation, or “globules”. PiZ mice that received four biweekly intravenous (IV) doses of 8 mg/kg AAT RNAi agent with 8 mg/kg of MLP delivery polymer over the course of 8 weeks showed a decrease in intracellular AAT globules compared to PiZ mice receiving saline or a Luciferase double-stranded siRNA control: dTCfgAfaGfU_{UNA}AfcUfcAfgCfgUfaAfgdTsdT (SEQ ID NO: 7); (Chol-TEG)uAuCfuUfaCfgCfuGfaGfuAfcUfuCfgAf(invdT) (SEQ ID NO: 8). The number of globules, the size of the globules and the area of the liver covered by globules was digitally quantitated from liver specimens stained with PAS-D. Mice treated with AAT RNAi agent had 85% fewer globules, 85% smaller globules, and 96% less area of the liver covered with globules than saline-injected controls (FIG. 3).

### Example 5. Analysis of Soluble and Insoluble Z-hAAT Protein in PiZ Mouse Liver Tissue

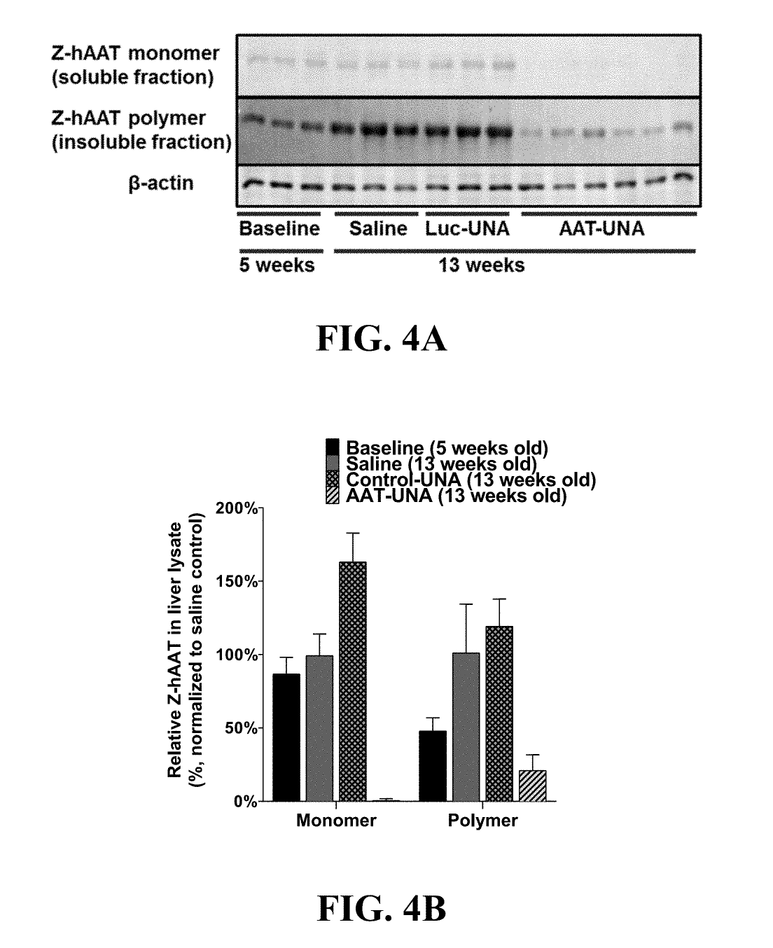

Homogenized liver tissue from PiZ mice treated with Duplex Pair SEQ ID NO: 5/6 was further analyzed to determine if both soluble Z-hAAT, expected to be mostly monomeric protein, and insoluble polymers of Z-hAAT were effectively reduced. A modified western blot protocol was used to separate the soluble and insoluble Z-hAAT fractions under non-denaturing conditions as previously described (Mueller et al. Molecular Therapy 2012). PiZ mice given four biweekly intravenous (IV) doses of 8 mg/kg Duplex Pair SEQ ID NO: 5/6 with 8 mg/kg of MLP delivery polymer for 8 weeks showed a >99% reduction in soluble and 79% reduction in insoluble Z-hAAT, compared to PiZ mice given four biweekly intravenous (IV) doses of saline (FIGS. 4A and 4B).

TABLE 9
Average levels of soluble and insoluble Z-hAAT protein in liver
lysate of male PiZ mice normalized to saline-injected mice

| | | Soluble | Insoluble polymer |
|---|---|---|---|
| | Number | (normalized to | (normalized to saline |
| Treatment | animals | saline control) | control) |
| Baseline (5 weeks old) | 6 | 0.866 ± 0.105 | 0.478 ± 0.083 |
| Saline (13 weeks old) | 7 | 0.992 ± 0.138 | 1.010 ± 0.309 |
| Control (SEQ ID 7/8) | 3 | 1.630 ± 0.162 | 1.192 ± 0.152 |
| (13 weeks old) | | | |
| SEQ ID 5/6 (13 weeks | 10 | 0.004 ± 0.013 | 0.209 ± 0.103 |
| old) | | | |

### Example 6. In Vivo Duration of Response from Single Injection of AAT Expression-Inhibiting Oligomeric Compounds in PiZ Mice

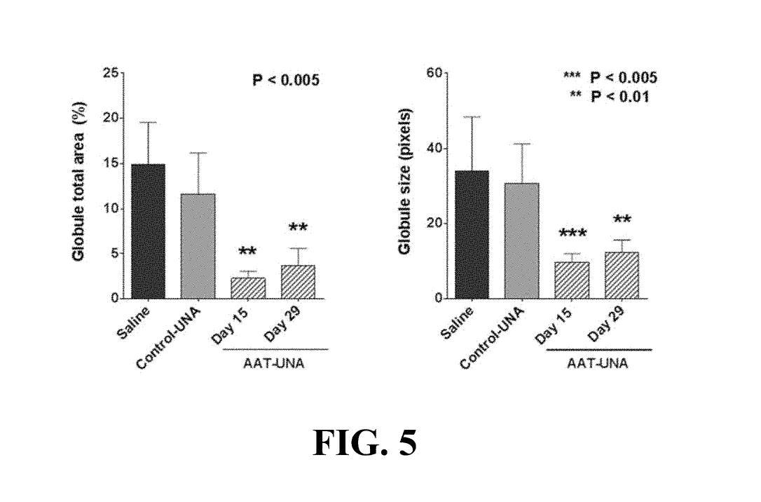

A single IV dose of saline, 8 mg/kg Control+8 mg/kg MLP delivery polymer, or 8 mg/kg Duplex Pair SEQ ID NO: 5/6 (AAT RNAi agent)+8 mg/kg MLP delivery polymer was administered to 6 month old female PiZ mice. Human AAT protein levels in serum were monitored for 29 days. At the indicated times, blood samples were collected and assayed for hAAT by ELISA. Day 1 samples were collected prior to trigger administration. For mRNA analysis, 3-4 mice were euthanized at each of days 3, 8, 15, 22, and 29. For euthanized mice, cardiac stick were performed for serum isolation for AAT ELISA (200 μl serum). Half of the left lateral liver lobe was collected and snap-freeze in liquid nitrogen for RNA isolation. The remainder of the left lobes were embedded into paraffin blocks for PAS-D staining with hematoxylin as counter-stain. Serum hAAT levels in mice given AAT Duplex Pair SEQ ID NO: 5/6 were 95% reduced on day 8 and remained reduced to day 29, at which time they were 79% reduced. Mice given AAT Duplex Pair SEQ ID NO: 5/6 were euthanized at either day 3, 8, 15, 22 or 29. Mice given saline or Luciferase siRNA control (SEQ ID 7/8) were euthanized on day 29. Levels of hAAT mRNA in the livers were measured by RT-qPCR. The hAAT mRNA in mice given AAT Duplex Pair SEQ ID NO: 5/6 was reduced by 97% on day 3 and remained reduced on day 29, at which time levels were 56% reduced. The size of the globules and the area of the liver covered by globules was digitally quantitated from liver specimens stained with PAS-D. Duplex Pair SEQ ID NO: 5/6 treated mice had 70% smaller globules at day 15 and 62% smaller globules at day 29. The area of the liver covered with globules was 83% reduced on day 15 and 72% reduced on day 29 (FIG. 5).

TABLE 10
Serum hAAT levels in PiZ mice following administration of one injection of saline,
Control Duplex Pair SEQ ID NO: 7/8, or Duplex Pair SEQ ID NO: 5/6 (AAT RNAi agent).

| Day | | Serum hAAT normalized to day 1 and controls | | | | | | |
|---|---|---|---|---|---|---|---|---|
| euthanized | Treatment | Day −2 | Day 1 | Day 3 | Day 8 | Day 15 | Day 22 | Day 29 |
| Day 29 | Saline | 1.000 ± 0.239 | 1.000 | 1.000 ± 0.368 | 1.000 ± 0.235 | 1.000 ± 0.097 | 1.000 ± 0.177 | 1.000 ± 0.272 |
| Day 29 | 8 mg/kg MLP | 1.268 ± 0.143 | 1.000 | 0.994 ± 0.153 | 1.090 ± 0.092 | 0.890 ± 0.080 | 1.171 ± 0.095 | 0.797 ± 0.074 |
| | delivery polymer + | | | | | | | |
| | 8 mg/kg Control | | | | | | | |
| Day 3 | 8 mg/kg MLP | 1.068 ± 0.070 | 1.000 | 0.255 ± 0.040 | — | — | — | — |
| | delivery polymer + | | | | | | | |
| Day 8 | 8 mg/kg AAT | 0.895 ± 0.129 | 1.000 | 0.184 ± 0.025 | 0.054 ± 0.004 | — | — | — |
| | Duplex Pair SEQ | | | | | | | |
| Day 15 | ID NO: 5/6 | 0.66 ± 0.121 | 1.000 | 0.243 ± 0.060 | 0.066 ± 0.023 | 0.095 ± 0.063 | — | — |
| Day 22 | | 0.779 ± 0.280 | 1.000 | 0.202 ± 0.054 | 0.044 ± 0.010 | 0.056 ± 0.011 | 0.100 ± 0.039 | — |
| Day 29 | | 0.653 ± 0.102 | 1.000 | 0.238 ± 0.062 | 0.052 ± 0.015 | 0.057 ± 0.016 | 0.103 ± 0.032 | 0.209 ± 0.071 |

TABLE 11
Relative hAAT mRNA levels in PiZ mice following administration
of one injection of saline, Control Duplex Pair SEQ ID NO: 7/8,
or Duplex Pair SEQ ID NO: 5/6 (AAT RNAi agent).

| | | Average | | |
|---|---|---|---|---|
| | | relative | Low | High |
| Treatment | Day | mRNA level | variance | variance |
| Saline | 29 | 1.000 | 0.072 | 0.078 |
| 8 mg/kg Luc-siRNA control + | 29 | 1.031 | 0.090 | 0.098 |
| 8 mg/kg MLP delivery polymer | | | | |
| 8 mg/kg Duplex Pair SEQ ID | 3 | 0.030 | 0.007 | 0.009 |
| 5/6 + 8 mg/kg MLP delivery | 8 | 0.032 | 0.014 | 0.024 |
| polymer | 15 | 0.158 | 0.060 | 0.096 |
| | 22 | 0.221 | 0.033 | 0.038 |
| | 29 | 0.439 | 0.057 | 0.066 |

### Example 7. Alpha-1 Antitrypsin (AAT) Knockdown in Primates Following AAT Expression-Inhibiting Oligomeric Compound Delivery

MLP delivery polymer and Duplex Pair SEQ ID NO: 5/6 (AAT RNAi agent) were made and combined in a pharmaceutically acceptable buffer. On day 1, cynomolgus macaque (Macaca fascicularis) primates (male and female, 3 to 9 kg) were co-injected with MLP delivery polymer and Duplex Pair SEQ ID NO: 5/6 at different dose combinations. The dose combinations injected were: 2.0 mg/kg MLP delivery polymer+4.0 AAT RNAi agent (n=3), 3 mg/kg MLP delivery polymer+1.5 mg/kg AAT RNAi agent (n=2), 3.0 mg/kg MLP delivery polymer+3.0 mg/kg AAT RNAi agent (n=3), 3.0 mg/kg MLP delivery polymer+6.0 mg/kg AAT RNAi agent (n=2), 6.0 mg/kg MLP delivery polymer+12 mg/kg AAT RNAi trigger (n=3) (0.050 s conversion factor used to determine RNAi agent concentration) and 12 mg/kg MLP delivery polymer+6.0 mg/kg AAT RNAi agent (n=12). For each injection the MLP delivery polymer+AAT RNAi agent (2 ml/kg) was injected into the saphenous vein using a 22 to 25 gauge intravenous catheter. At the indicated time points, blood samples were drawn and analyzed for AAT and toxicity markers. Blood was collected from the femoral vein and primates were fasted overnight before all blood collections. Blood tests for blood urea nitrogen (BUN), alanine transaminase (ALT), aspartate aminotransferase (AST), and creatinine were performed on an automated chemistry analyzer at Meriter laboratories or BASi. AAT levels were determined on a Cobas Integra 400 (Roche Diagnostics) according to the manufacturer's recommendations. Significant knockdown of AAT was observed at all dose combinations.

TABLE 12
Percent AAT Knockdown in NHPs

| MLP | SEQ ID 5/6 | | Day | | | | | | | | | | | | |
|---|---|---|---|---|---|---|---|---|---|---|---|---|---|---|---|
| (mg/kg) | (mg/kg) | Pretest | 2 | 3 | 8 | 11 | 15 | 22 | 26 | 29 | 33 | 36 | 43 | 47 | 50 |
| 2.0 | 4.0 | 0 | 11 | 27 | 63 | 73 | 81 | 85 | — | 80 | — | | | | |
| 3.0 | 1.5 | 0 | 16 | 30 | 59 | 70 | 74 | 76 | — | 74 | — | 70 | 63 | — | 51 |
| 3.0 | 3.0 | 0 | 15 | 29 | 63 | 74 | 82 | 88 | — | 85 | — | | | | |
| 3.0 | 6.0 | 0 | 10 | 25 | 63 | 74 | 82 | | | | | | | | |
| 6.0 | 12.0 | 0 | 18 | 30 | 61 | | | | | | | | | | |
| 12.0 | 6.0 | 0 | 3 | 19 | 60 | 84 | 88 | — | 91 | — | 86 | — | — | 76 | |

### Example 8. Repeat Administration of AAT Expression-Inhibiting Oligomeric Compounds

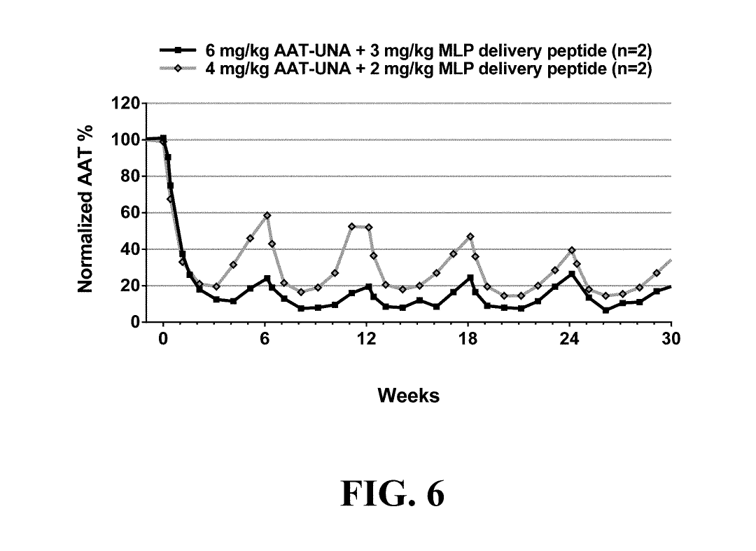

Cynomolgus macaque primates were given five doses of AAT RNAi trigger+MLP delivery polymer at six week intervals. Each dose contained MLP delivery polymer and Duplex Pair SEQ ID NO: 5/6 (AAT RNAi agent) at a 1:2 weight to weight ratio of the MLP to RNAi agent. The first injection was on day 1. The dose combinations injected were: 2.0 mg/kg MLP delivery polymer+4.0 AAT RNAi agent (n=2) and 3 mg/kg MLP delivery polymer+6 mg/kg AAT RNAi agent (n=2). Blood was collected at intervals throughout the study and AAT levels were measured from the serum as described. Repeat dosing at six week intervals reduced serum AAT levels by approximately 80-90% from two to thirty weeks after the first treatment of 3 mg/kg MLP delivery polymer+6 mg/kg AAT RNAi agent in the primates. Serum AAT was reduced by 80% following the first treatment of primates with 2.0 mg/kg MLP delivery polymer+4.0 AAT RNAi agent and by 85% following the fourth treatment. Serum AAT levels measured six weeks after each treatment with 2.0 mg/kg MLP delivery polymer+4.0 AAT RNAi agent rebounded less with each additional treatment (FIG. 6).

### Example 9. Treatment of PiZ Mice with AAT Expression-Inhibiting Oligomeric Compounds

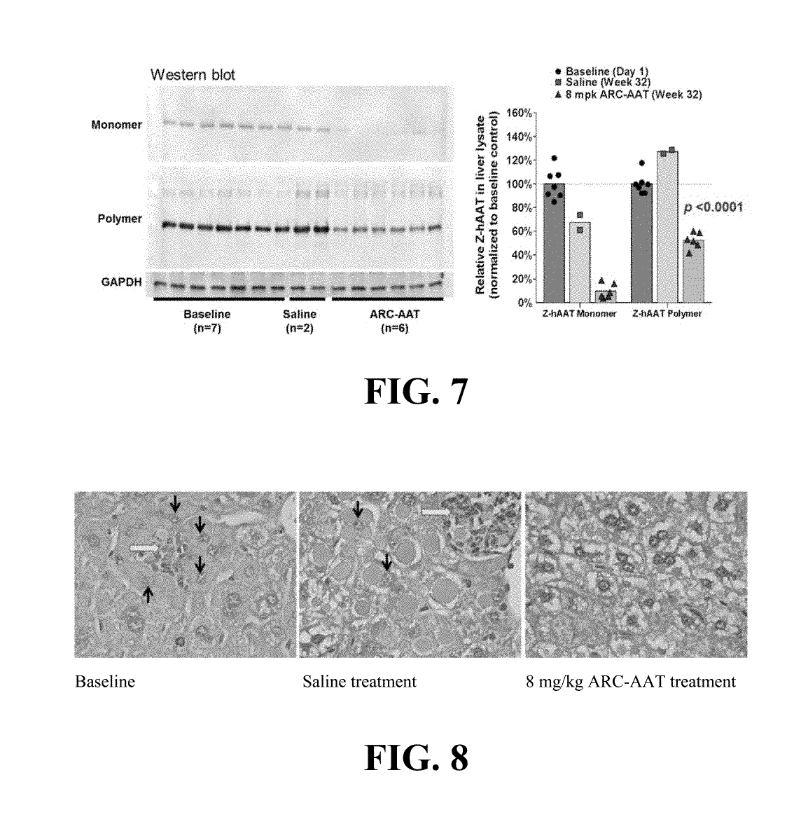

Male PiZ mice 11-17 weeks old at the start of the study that had already accumulated Z-hAAT globules were selected for analysis. These mice were treated every other week (biweekly) with Duplex Pair SEQ ID NO: 5/6 (AAT RNAi agent) at doses of 4, 8 or 12 mg/kg with MLP delivery polymer (dosed at 50% of the dose of AAT RNAi agent; i.e., a 2:1 ratio of AAT RNAi agent:MLP delivery polymer by weight) for 16, 24 or 32-33 weeks. Mice treated with AAT RNAi agent showed a reduction in the Z-AAT polymer burden and a reduction in the diseased liver morphology. (See FIGS. 7 and 8).

PiZ mice treated biweekly with 8 mg/kg AAT RNAi agent for 32 weeks were compared with mice at start of study (baseline) and control mice injected for 32 weeks with saline vehicle. Monomeric and polymeric Z-hAAT protein in the livers of these mice were compared by semi-quantitative Western blotting (FIG. 7). As shown in the bar graph in FIG. 7, AAT RNAi agent treatment reduced both monomeric and polymeric Z-AAT protein. Monomeric Z-AAT was reduced by 90% and polymeric Z-AAT was reduced by 48% in the AAT RNAi agent treated mice (p<0.0001).

Liver histology was compared between mice at start of study (baseline), mice injected with Duplex Pair SEQ ID NO: 5/6 (AAT RNAi agent) and control mice injected at the same intervals with saline. Examples of the results are shown in FIG. 8 and Table 13. Baseline mice had significant globule accumulation with globules that were 5-10 microns in diameter, compressed nuclei, apoptosis and inflammatory cell infiltration. Saline treated mice had significant globule accumulation with globules that were 25-35 microns in diameter, compressed nuclei, and inflammatory cell infiltration. AAT RNAi agent treated mice showed improvement over control by having minimal to moderate globule accumulation with globules that were 7-10 microns in diameter, fewer compressed nuclei than either baseline or saline-treated mice, and no inflammatory infiltration.

The globule burden, the area of the liver covered with globules, and the number of nuclei compressed due to hepatocytes being loaded with globules were reduced in AAT RNAi agent treated mice, consistent with the measured reduction in Z-AAT polymer. Livers of AAT RNAi agent treated mice also contained fewer foci of inflammatory cells (FIG. 8 and Table 13). Improving liver health with AAT RNAi agent treatment appears to improve the capacity of hepatocytes of AATD individuals to undergo autophagy and thereby can result in increased turnover of the remaining globules.

TABLE 13
Liver histology following Duplex Pair SEQ ID NO: 5/6
(AAT RNAi agent) and MLP treatment of PiZ mice.

| | Score | | |
|---|---|---|---|
| | | SEQ ID NO: 5/6 | |
| Observation | | (AAT RNAi agent) + | |
| Overall globule | Baseline | MLP treated | Saline treated |
| burden | (n = 12) | (n = 16) | (n = 13) |
| Compressed nuclei | 7/12 | 3/16 | 13/13 |

Inflammatory foci

| | | | |
|---|---|---|---|
| None | 0 | 5 | 0 |
| Moderate | 12 | 11 | 0 |
| Severe | 0 | 0 | 13 |
| Total number | 12 | 16 | 13 |
| animals | | | |

### Example 10

Male PiZ mice 3-6 months old at start of study are dosed every other week (biweekly, Q2W), with 8 mg/kg Duplex Pair SEQ ID NO: 5/6 (AAT RNAi agent) and 4 mg/kg MLP. One week after the first AAT RNAi agent injection in which a reduction in Z-AAT production is exhibited, the PiZ mice are dosed with one 5 mg tablet fluphenazine under skin for 21 days slow release. The mice are sacrificed at 2 to 28 days after final AAT RNAi agent injection. Histological evaluation of the liver, liver PAS-D globule quantitation, and Z-AAT monomer and polymer measurements by Western blotting are examined.

### Example 11

Male PiZ mice 3-16 months old at start of study are dosed 2-16 times Q2W with 4 mg/kg to 12 mg/kg Duplex Pair SEQ ID NO: 5/6 (AAT RNAi agent) and MLP delivery polymer (dosed at 50% of the dose of AAT RNAi agent; i.e., a 2:1 ratio of AAT RNAi agent:MLP delivery polymer by weight). On the day of the first injection or one week thereafter, dose with one 5 mg tablet fluphenazine under skin for 21 days slow release. The mice are dosed once every third week (Q3W) for 1 to 11 doses. The mice are sacrificed at 2 to 28 days after final AAT RNAi agent injection. Histological evaluation of the liver, liver PAS-D globule quantitation, and Z-AAT monomer and polymer measurements by Western blotting are examined.

### Example 12

Male PiZ mice 3-16 months old at start of study are dosed 4-16 times Q2W with 4 mg/kg to 12 mg/kg Duplex Pair SEQ ID NO: 5/6 (AAT RNAi agent) and MLP delivery polymer (dosed at 50% of the dose of AAT RNAi agent; i.e., a 2:1 ratio of AAT RNAi agent:MLP delivery polymer by weight) to restore health. At 1, 2, 3, 4, 5, 6, 7, 8, 9, 10, 11, 12, 13, 14, 15, 16, 17, 18, 19, 20, 21, 22, 23, 24, 25, or 26 weeks after first injection, dose with one 5 mg tablet fluphenazine under skin for 21 days slow release. The mice are dosed Q3W for 1, 2, 3, 4, 5, 6, 7, 8, 9, 10 or 11 doses. The mice are sacrificed at 2 to 28 days after final AAT RNAi agent injection. Histological evaluation of the liver, liver PAS-D globule quantitation, and Z-AAT monomer and polymer measurements by Western blotting are examined.

### Example 13

Male PiZ mice 3-16 months old at start of study are dosed 4-16 times Q2W with 4 mg/kg to 12 mg/kg Duplex Pair SEQ ID NO: 5/6 (AAT RNAi agent) and MLP delivery polymer (dosed at 50% of the dose of AAT RNAi agent; i.e., a 2:1 ratio of AAT RNAi agent:MLP delivery polymer by weight) to restore health. At 1, 2, 3, 4, 5, 6, 7, 8, 9, 10, 11, 12, 13, 14, 15, 16, 17, 18, 19, 20, 21, 22, 23, 24, 25, or 26 weeks after first AAT RNAi agent injection, begin dosing with any of the autophagy enhancing agents in Table 4. Dose with autophagy enhancing agent for 1 to 30 weeks. The mice are sacrificed at 2 to 28 days after final AAT RNAi agent injection. Histological evaluation of the liver, liver PAS-D globule quantitation, and Z-AAT monomer and polymer measurements by Western blotting are examined.

Reduction in Z-AAT polymer, globule burden, the area of the liver covered with globules, and the number of nuclei compressed due to hepatocytes being loaded with globules are expected in mice administered both AAT RNAi agent and a selected autophagy enhancing agent as compared to mice treated solely with AAT RNAi agent.

### Example 14. Treatment of PiZ Mice with AAT Expression-Inhibiting Oligomeric Compounds

Male PiZ mice 15-21 weeks old at the start of the study were selected for study. Mice were divided into six groups for treatment for up to 42 weeks as follows:

TABLE 14
Example 14 treatment protocol.

| | | | Animals |
|---|---|---|---|
| Group | Treatment(s) and Dosing Regimen | Euthanization | (n =) |
| 1 | Baseline | Day 1 | 6 |
| 2 | Saline: 20 × q2w multiple injections; | Week 39 | 5 |
| | last injection week 38 | | |
| 3 | Saline: 20 × q2w multiple injections; last injection | Week 39 | 6 |
| | week 38 | | |
| | Fluphenazine: 5 mg tablet at week 36 | | |
| 4 | 8 mg/kg Duplex Pair SEQ ID NO: 5/6 (AAT RNAi | Week 39 | 8 |
| | agent) + 4 mg/kg MLP: (20 × q2w multiple | | |
| | injections; last injection week 38) | | |
| 5 | 8 mg/kg Duplex Pair SEQ ID NO: 5/6 (AAT RNAi | Week 39 | 7 |
| | agent) + 4 mg/kg MLP: (20 × q2w multiple | | |
| | injections; last injection week 38) | | |
| | Fluphenazine: 5 mg tablet at week 36 | | |
| 6 | 8 mg/kg Duplex Pair SEQ ID NO: 5/6 (AAT RNAi | Week 42 | 7 |
| | agent) + 4 mg/kg MLP: (21 × q2w multiple | | |
| | injections; last injection week 40) | | |
| | Fluphenazine: 5 mg tablet at week 36 and week 39 | | |

One animal from each of Groups 2, 3, and 4 died prior to completion of the study. After sacrifice, monomeric and polymeric Z-hAAT protein in the livers of these mice were compared by semi-quantitative Western blotting over a course of 3 separate runs. Western blot Run 1 included six mice from Group 1, four mice from Group 2, and five mice from Group 4. Western blot Run 2 includes three mice from Group 1, six mice from Group 3 and 5 mice from Group 5. Western blot Run 3 includes three mice from Group 1, 2 mice from Group 4, 2 mice from Group 5, 7 mice from Group 6.

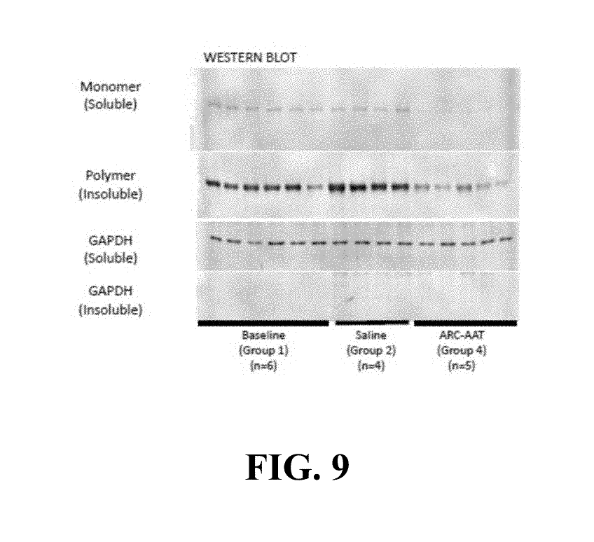

FIG. 9 shows Western blotting for some of the animals from Groups 1, 2, and 4 (Run 1). Treatment of mice in Group 4 (AAT RNAi agent) reduced both monomeric and polymeric Z-AAT protein compared to Group 1 (baseline) and Group 2 (saline), as shown by semi-quantitative analysis of Western Blot for Run 1:

TABLE 15
Z-hAAT monomeric protein derived from semi-quantitative
Western blot analysis of Run 1.

| | | Average | Average |
|---|---|---|---|
| | | Normalized to | Normalized |
| | | Baseline | to Saline |
| Group (n) | Average (Standard deviation) | (Group 1) | (Group 2) |
| 1 (n = 6) | 566.6 (34.6) | 1.000 | 1.208 |
| 2 (n = 4) | 469.2 (63.5) | 0.828 | 1.000 |
| 4 (n = 5) | 71.4 (15.6) | 0.126 | 0.152 |

TABLE 16
Z-hAAT polymeric protein derived from semi-quantitative
Western blot analysis of Run 1.

| | | Average | Average |
|---|---|---|---|
| | | Normalized to | Normalized |
| | | Baseline | to Saline |
| Group (n) | Average (Standard deviation) | (Group 1) | (Group 2) |
| 1 (n = 6) | 4322.9 (1259.2) | 1.000 | 0.569 |
| 2 (n = 4) | 7594.7 (916.4) | 1.757 | 1.000 |
| 4 (n = 5) | 1809.1 (932.5) | 0.419 | 0.238 |

It is anticipated that at least some animals treated in Groups 5 and/or 6 will exhibit a reduction in the level of polymeric Z-hAAT protein and/or monomeric Z-hAAT protein as compared to animals in treatment Group 3 (autophagy enhancing agent only) and Group 4 (AAT expression-inhibiting oligomeric compound only).

1. A method for the treatment of a condition, manifestation, or disease caused by alpha-1 antitrypsin deficiency (AATD), the method comprising administering to a subject in need thereof a composition comprising an effective amount of an AAT expression-inhibiting oligomeric compound and a composition comprising an effective amount of an autophagy enhancing agent.

2. The method of claim 1, wherein the method treats, prevents, or manages a pathological condition or disease caused by AATD.

3. The method of claim 2, wherein the pathological condition or disease caused by alpha-1 antitrypsin deficiency is chronic hepatitis, cirrhosis, hepatocellular carcinoma, or fulminant hepatic failure.

4. The method of claim 1, wherein the method further comprises the administration of a second AAT expression-inhibiting oligomeric compound.

5. The method of claim 1, wherein the expression-inhibiting oligomeric compound has a nucleobase sequence that contains at least one modified nucleotide or modified internucleoside linkage.

6. The method of claim 5, wherein the modified nucleotide is selected from the group consisting of: 2′-O-methyl modified nucleotide, 2′-deoxy-2′-fluoro modified nucleotide, 2′-deoxy-modified nucleotide, locked nucleotide, abasic nucleotide, deoxythymidine, inverted deoxythymidine, 2′-amino-modified nucleotide, 2′-alkyl-modified nucleotide, morpholino nucleotide, and non-natural base comprising nucleotide.

7. The method of claim 5, wherein the modified internucleoside linkage is a phosphorothioate linkage.

8. The method of claim 1, wherein the AAT expression-inhibiting oligomeric compound is a double-stranded RNAi agent.

9. The method of claim 1, wherein the autophagy enhancing agent is selected from the group consisting of: ezetimibe, carbamazepine, oxcarbazepine, verapamil, loperamide, nimodipine, nitrendipine, niguldipine, amiodarone, clonidine, rilmenidine, lithium, valproic acid, fluphenazine, calpastatin, pimozide, fluspirilene, glyburide, metformin, rapamycin, minoxidil, trehalose, beclin 1 peptide, a statin, an m-Tor inhibitor, and a tyrosine kinase inhibitor.

10. A method for the treatment of a condition, manifestation, or disease caused by alpha-1 antitrypsin deficiency (AATD), the method comprising administering to a subject in need thereof a composition comprising an effective amount of an AAT expression-inhibiting oligomeric compound and a composition comprising an effective amount of a bile acid derivative.

11. The method of claim 10, wherein the bile acid derivative is ursodeoxcholic acid or nor-ursodeoxycholic acid.

12. The method of claim 1, wherein the AAT expression-inhibiting oligomeric compound is conjugated, directly or indirectly, to a targeting moiety.

13. The method of claim 10, wherein the AAT expression-inhibiting oligomeric compound is conjugated, directly or indirectly, to a targeting moiety.

14. The method of claim 1, wherein the AAT expression-inhibiting oligomeric compound is administered separately from the autophagy enhancing agent.

15. The method of claim 10, wherein the AAT expression-inhibiting oligomeric compound is administered separately from the bile acid derivative.

16. The method of claim 1, wherein at least one of the count, size, or area percentage of Z-AAT globules retained in the cell, tissue, or organism, is reduced.

17. The method of claim 10, wherein at least one of the count, size, or area percentage of Z-AAT globules retained in the cell, tissue, or organism, is reduced compared to pre-treatment levels.

18. A method for reducing Z-AAT globule burden in a liver cell, cell, tissue, or organism, the method comprising administering to the liver cell, cell, tissue, or organism, an effective amount of an AAT expression-inhibiting oligomeric compound, or a composition comprising same, and an effective amount of either (i) an autophagy enhancing agent, or a composition comprising same, or (ii) a bile acid derivative, or a composition comprising same.

19. The method of claim 18, wherein the AAT expression-inhibiting oligomeric compound, and the autophagy enhancing agent or bile acid derivative, are administered separately.

20. The method of claim 18, wherein the count, size, and/or area percentage of Z-AAT globules retained in the liver cell, cell, tissue, or organism, is reduced compared to pre-treatment levels.

21. A composition for the treatment of a condition, manifestation, or disease caused by alpha-1 antitrypsin deficiency (AATD), comprising an AAT expression-inhibiting oligomeric compound and either (i) an autophagy enhancing agent, or (ii) a bile acid derivative.


SEQUENCE LISTING (8 sequences)

SEQ ID NO: 1 - RNA, 18 nt, Artificial Sequence
  antisense strand of dsRNA
ggaacuuggugaugauau

SEQ ID NO: 2 - RNA, 18 nt, Artificial Sequence
  sense strand of dsRNA
auaucaucaccaaguucc

SEQ ID NO: 3 - DNA, 21 bp, Artificial Sequence
  antisense strand of dsRNA
  modified_base (2)..(19): /mod_base = “2′-hydroxy corresponding nucleoside”
tggaacuuggugaugauautt

SEQ ID NO: 4 - DNA, 23 bp, Artificial Sequence
  sense strand of dsRNA
  modified_base (1)..(22): /mod_base = “2′-hydroxy corresponding nucleoside”
uauauaucaucaccaaguuccat

SEQ ID NO: 5 - DNA, 21 bp, Artificial Sequence
  antisense strand of dsRNA
  modified_base 7: /mod_base = “2′-3′ seco corresponding nucleotide”
  modified_base 2,4,6,8,10,12,14,16,18: /mod_base = “2′-deoxy-2′-fluoro corresponding nucleoside”
  modified_base 3,5,9,11,13,15,17,19: /mod_base = “2′-O-methyl corresponding nucleoside”
  modified_base 21: /mod_base = “5′-phosphorothioate corresponding nucleotide”
tggaacuuggugaugauautt

SEQ ID NO: 6 - DNA, 23 bp, Artificial Sequence
  sense strand of dsRNA
  modified_base 2: /mod_base = “2′-hydroxy corresponding nucleoside”
  modified_base 4,6,8,10,12,14,16,18,20,22: /mod_base = “2′-deoxy-2′-fluoro corresponding nucleoside”
  modified_base 1,3,5,7,9,11,13,15,17,19,21: /mod_base = “2′-O-methyl corresponding nucleoside”
  modified_base 23: /mod_base = “3′-3′-linked deoxythymidine”
uauauaucaucaccaaguuccat

SEQ ID NO: 7 - DNA, 21 bp, Artificial Sequence
  antisense strand of dsRNA
  modified_base 7: /mod_base = “2′-3′ seco corresponding nucleotide”
  modified_base 2,4,6,8,10,12,14,16,18: /mod_base = “2′-deoxy-2′-fluoro corresponding nucleoside”
  modified_base 3,5,9,11,13,15,17,19: /mod_base = “2′-O-methyl corresponding nucleoside”
  modified_base 21: /mod_base = “5′-phosphorothioate corresponding nucleotide”
tcgaaguacucagcguaagtt

SEQ ID NO: 8 - DNA, 23 bp, Artificial Sequence
  sense strand of dsRNA
  modified_base 2: /mod_base = “2′-hydroxy corresponding nucleoside”
  modified_base 4,6,8,10,12,14,16,18,20,22: /mod_base = “2′-deoxy-2′-fluoro corresponding nucleoside”
  modified_base 1,3,5,7,9,11,13,15,17,19,21: /mod_base = “2′-O-methyl corresponding nucleoside”
  modified_base 23: /mod_base = “3′-3′-linked deoxythymidine”
uaucuuacgcugaguacuucgat